SEQUENCE LISTING (15 sequences)

SEQ ID NO: 1 - protein, 458 aa, Artificial Sequence
  Synthetic polypeptide
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQIAKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 2 - DNA, 1377 bp, Artificial Sequence
  Synthetic polynucleotide
atggagaataagacagaaacaaccgtaagacggaggcggaggattatcttgttccctgtaccatttcagggccatattaatccgatcctccaattagcaaacgtcctctactccaagggattttcaataacaatcttccatactaactttaacaagcctaaaacgagtaattatcctcactttacattcaggttcattctagacaacgaccctcaggatgagcgtatctcaaatttacctacgcatggccccttggcaggtatgcgaataccaataatcaatgagcatggagccgatgaactccgtcgcgagttagagcttctcatgctcgcaagtgaggaagacgaggaagtttcgtgcctaataactgatgcgctttggtacttcgcccaatcagtcgcagactcactgaatctacgccgtttggtccttatgacaagttcattattcaactttcacgcacatgtatcactgccgcaatttgacgagttgggttacctggacccggatgacaaaacgcgattggaggaacaagcgtcgggcttccccatgctgaaagtcaaagatattaagagcgcttatagtaattggcaaattgcgaaagaaattctcggaaaaatgataaagcaaaccaaagcgtcctctggagtaatctggaactccttcaaggagttagaggaatctgaacttgaaacggtcatcagagaaatccccgctccctcgttcttaattccactacccaagcaccttactgcaagtagcagttccctcctagatcatgaccgaaccgtgtttcagtggctggatcagcaacccccgtcgtcagttctatatgtaagctttgggagtacttcggaagtggatgaaaaggacttcttagagattgcgcgagggctcgtggatagcaaacagagcttcctgtgggtagtgagaccgggattcgttaagggctcgacgtgggtcgagccgttgccagatggttttctaggggagagagggagaatcgtgaaatgggttccacagcaagaggttttggctcacggagctataggggccttttggacccactctggttggaattctactcttgaaagtgtctgtgaaggcgttccaatgatattttctgattttgggcttgaccagcctctaaacgctcgctatatgtctgatgtgttgaaggttggcgtgtacctggagaatggttgggaaaggggggaaattgccaacgccatacgccgggtaatggtggacgaggaaggtgagtacatacgtcagaacgctcgggttttaaaacaaaaagcggacgtcagccttatgaagggaggtagctcctatgaatccctagaatccttggtaagctatatatcttcgttataa

SEQ ID NO: 3 - protein, 458 aa, Artificial Sequence
  Synthetic polypeptide
MNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSLENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYS

SEQ ID NO: 4 - DNA, 1377 bp, Artificial Sequence
  Synthetic polynucleotide
atgaactggcaaatcctgaaagaaatcctgggtaaaatgatcaaacaaaccaaagcgtcgtcgggcgttatctggaactccttcaaagaactggaagaatcagaactggaaaccgttattcgcgaaatcccggctccgtcgttcctgattccgctgccgaaacatctgaccgcgagcagcagcagcctgctggatcacgaccgtacggtctttcagtggctggatcagcaaccgccgtcatcggtgctgtatgtttcattcggtagcacctctgaagtcgatgaaaaagactttctggaaatcgctcgcggcctggtggatagtaaacagtccttcctgtgggtggttcgtccgggttttgtgaaaggcagcacgtgggttgaaccgctgccggatggcttcctgggtgaacgcggccgtattgtcaaatgggtgccgcagcaagaagtgctggcacatggtgctatcggcgcgttttggacccactctggttggaacagtacgctggaatccgtttgcgaaggtgtcccgatgattttcagcgattttggcctggaccagccgctgaatgcccgctatatgtctgatgttctgaaagtcggtgtgtacctggaaaacggttgggaacgtggcgaaattgcgaatgccatccgtcgcgttatggtcgatgaagaaggcgaatacattcgccagaacgctcgtgtcctgaaacaaaaagcggacgtgagcctgatgaaaggcggtagctcttatgaatcactggaatcgctggttagctacatcagttccctggaaaataaaaccgaaaccacggtgcgtcgccgtcgccgtattatcctgttcccggttccgtttcagggtcatattaacccgatcctgcaactggcgaatgttctgtattcaaaaggcttttcgatcaccatcttccatacgaacttcaacaaaccgaaaaccagtaactacccgcactttacgttccgctttattctggataacgacccgcaggatgaacgtatctccaatctgccgacccacggcccgctggccggtatgcgcattccgattatcaatgaacacggtgcagatgaactgcgccgtgaactggaactgctgatgctggccagtgaagaagatgaagaagtgtcctgtctgatcaccgacgcactgtggtatttcgcccagagcgttgcagattctctgaacctgcgccgtctggtcctgatgacgtcatcgctgttcaattttcatgcgcacgtttctctgccgcaatttgatgaactgggctacctggacccggatgacaaaacccgtctggaagaacaagccagtggttttccgatgctgaaagtcaaagacattaaatccgcctattcgtaa

SEQ ID NO: 5 - protein, 475 aa, Artificial Sequence
  Synthetic polypeptide
MNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSLYKDDSGYSSSYAAAAGMENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYS

SEQ ID NO: 6 - DNA, 1428 bp, Artificial Sequence
  Synthetic polynucleotide
atgaactggcaaatcctgaaagaaatcctgggtaaaatgatcaaacaaaccaaagcgtcgtcgggcgttatctggaactccttcaaagaactggaagaatcagaactggaaaccgttattcgcgaaatcccggctccgtcgttcctgattccgctgccgaaacatctgaccgcgagcagcagcagcctgctggatcacgaccgtacggtctttcagtggctggatcagcaaccgccgtcatcggtgctgtatgtttcattcggtagcacctctgaagtcgatgaaaaagactttctggaaatcgctcgcggcctggtggatagtaaacagtccttcctgtgggtggttcgtccgggttttgtgaaaggcagcacgtgggttgaaccgctgccggatggcttcctgggtgaacgcggccgtattgtcaaatgggtgccgcagcaagaagtgctggcacatggtgctatcggcgcgttttggacccactctggttggaacagtacgctggaatccgtttgcgaaggtgtcccgatgattttcagcgattttggcctggaccagccgctgaatgcccgctatatgtctgatgttctgaaagtcggtgtgtacctggaaaacggttgggaacgtggcgaaattgcgaatgccatccgtcgcgttatggtcgatgaagaaggcgaatacattcgccagaacgctcgtgtcctgaaacaaaaagcggacgtgagcctgatgaaaggcggtagctcttatgaatcactggaatcgctggttagctacatcagttccctgtacaaagatgacagcggttatagcagcagctatgcggcggcggcgggtatggaaaataaaaccgaaaccacggtgcgtcgccgtcgccgtattatcctgttcccggttccgtttcagggtcatattaacccgatcctgcaactggcgaatgttctgtattcaaaaggcttttcgatcaccatcttccatacgaacttcaacaaaccgaaaaccagtaactacccgcactttacgttccgctttattctggataacgacccgcaggatgaacgtatctccaatctgccgacccacggcccgctggccggtatgcgcattccgattatcaatgaacacggtgcagatgaactgcgccgtgaactggaactgctgatgctggccagtgaagaagatgaagaagtgtcctgtctgatcaccgacgcactgtggtatttcgcccagagcgttgcagattctctgaacctgcgccgtctggtcctgatgacgtcatcgctgttcaattttcatgcgcacgtttctctgccgcaatttgatgaactgggctacctggacccggatgacaaaacccgtctggaagaacaagccagtggttttccgatgctgaaagtcaaagacattaaatccgcctattcgtaa

SEQ ID NO: 7 - protein, 1268 aa, Artificial Sequence
  Synthetic polypeptide
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSLGSGANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD

SEQ ID NO: 8 - DNA, 3807 bp, Artificial Sequence
  Synthetic polynucleotide
atggagaataagacagaaacaaccgtaagacggaggcggaggattatcttgttccctgtaccatttcagggccatattaatccgatcctccaattagcaaacgtcctctactccaagggattttcaataacaatcttccatactaactttaacaagcctaaaacgagtaattatcctcactttacattcaggttcattctagacaacgaccctcaggatgagcgtatctcaaatttacctacgcatggccccttggcaggtatgcgaataccaataatcaatgagcatggagccgatgaactccgtcgcgagttagagcttctcatgctcgcaagtgaggaagacgaggaagtttcgtgcctaataactgatgcgctttggtacttcgcccaatcagtcgcagactcactgaatctacgccgtttggtccttatgacaagttcattattcaactttcacgcacatgtatcactgccgcaatttgacgagttgggttacctggacccggatgacaaaacgcgattggaggaacaagcgtcgggcttccccatgctgaaagtcaaagatattaagagcgcttatagtaattggcaaattctgaaagaaattctcggaaaaatgataaagcaaaccaaagcgtcctctggagtaatctggaactccttcaaggagttagaggaatctgaacttgaaacggtcatcagagaaatccccgctccctcgttcttaattccactacccaagcaccttactgcaagtagcagttccctcctagatcatgaccgaaccgtgtttcagtggctggatcagcaacccccgtcgtcagttctatatgtaagctttgggagtacttcggaagtggatgaaaaggacttcttagagattgcgcgagggctcgtggatagcaaacagagcttcctgtgggtagtgagaccgggattcgttaagggctcgacgtgggtcgagccgttgccagatggttttctaggggagagagggagaatcgtgaaatgggttccacagcaagaggttttggctcacggagctataggggccttttggacccactctggttggaattctactcttgaaagtgtctgtgaaggcgttccaatgatattttctgattttgggcttgaccagcctctaaacgctcgctatatgtctgatgtgttgaaggttggcgtgtacctggagaatggttgggaaaggggggaaattgccaacgccatacgccgggtaatggtggacgaggaaggtgagtacatacgtcagaacgctcgggttttaaaacaaaaagcggacgtcagccttatgaagggaggtagctcctatgaatccctagaatccttggtaagctatatatcttcgttaggttctggtgcaaacgctgaacgtatgataacgcgcgtccacagccaacgtgagcgtttgaacgaaacgcttgtttctgagagaaacgaagtccttgccttgctttccagggttgaagccaaaggtaaaggtattttacaacaaaaccagatcattgctgaattcgaagctttgcctgaacaaacccggaagaaacttgaaggtggtcctttctttgaccttctcaaatccactcaggaagcaattgtgttgccaccatgggttgctctagctgtgaggccaaggcctggtgtttgggaatacttacgagtcaatctccatgctcttgtcgttgaagaactccaacctgctgagtttcttcatttcaaggaagaactcgttgatggagttaagaatggtaatttcactcttgagcttgatttcgagccattcaatgcgtctatccctcgtccaacactccacaaatacattggaaatggtgttgacttccttaaccgtcatttatcggctaagctcttccatgacaaggagagtttgcttccattgcttaagttccttcgtcttcacagccaccagggcaagaacctgatgttgagcgagaagattcagaacctcaacactctgcaacacaccttgaggaaagcagaagagtatctagcagagcttaagtccgaaacactgtatgaagagtttgaggccaagtttgaggagattggtcttgagaggggatggggagacaatgcagagcgtgtccttgacatgatacgtcttcttttggaccttcttgaggcgcctgatccttgcactcttgagacttttcttggaagagtaccaatggtgttcaacgttgtgatcctctctccacatggttactttgctcaggacaatgttcttggttaccctgacactggtggacaggttgtttacattcttgatcaagttcgtgctctggagatagagatgcttcaacgtattaagcaacaaggactcaacattaaaccaaggattctcattctaactcgacttctacctgatgcggtaggaactacatgcggtgaacgtctcgagagagtttatgattctgagtactgtgatattcttcgtgtgcccttcagaacagagaagggtattgttcgcaaatggatctcaaggttcgaagtctggccatatctagagacttacaccgaggatgctgcggttgagctatcgaaagaattgaatggcaagcctgaccttatcattggtaactacagtgatggaaatcttgttgcttctttattggctcacaaacttggtgtcactcagtgtaccattgctcatgctcttgagaaaacaaagtacccggattctgatatctactggaagaagcttgacgacaagtaccatttctcatgccagttcactgcggatattttcgcaatgaaccacactgatttcatcatcactagtactttccaagaaattgctggaagcaaagaaactgttgggcagtatgaaagccacacagcctttactcttcccggattgtatcgagttgttcacgggattgatgtgtttgatcccaagttcaacattgtctctcctggtgctgatatgagcatctacttcccttacacagaggagaagcgtagattgactaagttccactctgagatcgaggagctcctctacagcgatgttgagaacaaagagcacttatgtgtgctcaaggacaagaagaagccgattctcttcacaatggctaggcttgatcgtgtcaagaacttgtcaggtcttgttgagtggtacgggaagaacacccgcttgcgtgagctagctaacttggttgttgttggaggagacaggaggaaagagtcaaaggacaatgaagagaaagcagagatgaagaaaatgtatgatctcattgaggaatacaagctaaacggtcagttcaggtggatctcctctcagatggaccgggtaaggaacggtgagctgtaccggtacatctgtgacaccaagggtgcttttgtccaacctgcattatatgaagcctttgggttaactgttgtggaggctatgacttgtggtttaccgactttcgccacttgcaaaggtggtccagctgagatcattgtgcacggtaaatcgggtttccacattgacccttaccatggtgatcaggctgctgatactcttgctgatttcttcaccaagtgtaaggaggatccatctcactgggatgagatctcaaaaggagggcttcagaggattgaggagaaatacacttggcaaatctattcacagaggctcttgacattgactggtgtgtatggattctggaagcatgtctcgaaccttgaccgtcttgaggctcgccgttaccttgaaatgttctatgcattgaagtatcgcccattggctcaggctgttcctcttgcacaagatgattga

SEQ ID NO: 9 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 10 - DNA, 1377 bp, Stevia rebaudiana
atggagaataagacagaaacaaccgtaagacggaggcggaggattatcttgttccctgtaccatttcagggccatattaatccgatcctccaattagcaaacgtcctctactccaagggattttcaataacaatcttccatactaactttaacaagcctaaaacgagtaattatcctcactttacattcaggttcattctagacaacgaccctcaggatgagcgtatctcaaatttacctacgcatggccccttggcaggtatgcgaataccaataatcaatgagcatggagccgatgaactccgtcgcgagttagagcttctcatgctcgcaagtgaggaagacgaggaagtttcgtgcctaataactgatgcgctttggtacttcgcccaatcagtcgcagactcactgaatctacgccgtttggtccttatgacaagttcattattcaactttcacgcacatgtatcactgccgcaatttgacgagttgggttacctggacccggatgacaaaacgcgattggaggaacaagcgtcgggcttccccatgctgaaagtcaaagatattaagagcgcttatagtaattggcaaattctgaaagaaattctcggaaaaatgataaagcaaaccaaagcgtcctctggagtaatctggaactccttcaaggagttagaggaatctgaacttgaaacggtcatcagagaaatccccgctccctcgttcttaattccactacccaagcaccttactgcaagtagcagttccctcctagatcatgaccgaaccgtgtttcagtggctggatcagcaacccccgtcgtcagttctatatgtaagctttgggagtacttcggaagtggatgaaaaggacttcttagagattgcgcgagggctcgtggatagcaaacagagcttcctgtgggtagtgagaccgggattcgttaagggctcgacgtgggtcgagccgttgccagatggttttctaggggagagagggagaatcgtgaaatgggttccacagcaagaggttttggctcacggagctataggggccttttggacccactctggttggaattctactcttgaaagtgtctgtgaaggcgttccaatgatattttctgattttgggcttgaccagcctctaaacgctcgctatatgtctgatgtgttgaaggttggcgtgtacctggagaatggttgggaaaggggggaaattgccaacgccatacgccgggtaatggtggacgaggaaggtgagtacatacgtcagaacgctcgggttttaaaacaaaaagcggacgtcagccttatgaagggaggtagctcctatgaatccctagaatccttggtaagctatatatcttcgttataa

SEQ ID NO: 11 - protein, 808 aa, Arabidopsis thaliana
MANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD

SEQ ID NO: 12 - DNA, 2427 bp, Arabidopsis thaliana
atggcaaacgctgaacgtatgattacccgtgtccactcccaacgcgaacgcctgaacgaaaccctggtgtcggaacgcaacgaagttctggcactgctgagccgtgtggaagctaagggcaaaggtattctgcagcaaaaccagattatcgcggaatttgaagccctgccggaacaaacccgcaaaaagctggaaggcggtccgtttttcgatctgctgaaatctacgcaggaagcgatcgttctgccgccgtgggtcgcactggcagtgcgtccgcgtccgggcgtttgggaatatctgcgtgtcaacctgcatgcactggtggttgaagaactgcagccggctgaatttctgcacttcaaggaagaactggttgacggcgtcaaaaacggtaattttaccctggaactggattttgaaccgttcaatgccagtatcccgcgtccgacgctgcataaatatattggcaacggtgtggactttctgaatcgccatctgagcgcaaagctgttccacgataaagaatctctgctgccgctgctgaaattcctgcgtctgcatagtcaccagggcaagaacctgatgctgtccgaaaaaattcagaacctgaataccctgcaacacacgctgcgcaaggcggaagaatacctggccgaactgaaaagtgaaaccctgtacgaagaattcgaagcaaagttcgaagaaattggcctggaacgtggctggggtgacaatgctgaacgtgttctggatatgatccgtctgctgctggacctgctggaagcaccggacccgtgcaccctggaaacgtttctgggtcgcgtgccgatggttttcaacgtcgtgattctgtccccgcatggctattttgcacaggacaatgtgctgggttacccggataccggcggtcaggttgtctatattctggatcaagttcgtgcgctggaaattgaaatgctgcagcgcatcaagcagcaaggcctgaacatcaaaccgcgtattctgatcctgacccgtctgctgccggatgcagttggtaccacgtgcggtgaacgtctggaacgcgtctatgacagcgaatactgtgatattctgcgtgtcccgtttcgcaccgaaaagggtattgtgcgtaaatggatcagtcgcttcgaagtttggccgtatctggaaacctacacggaagatgcggccgtggaactgtccaaggaactgaatggcaaaccggacctgattatcggcaactatagcgatggtaatctggtcgcatctctgctggctcataaactgggtgtgacccagtgcacgattgcacacgctctggaaaagaccaaatatccggattcagacatctactggaaaaagctggatgacaaatatcatttttcgtgtcagttcaccgcggacatttttgccatgaaccacacggattttattatcaccagtacgttccaggaaatcgcgggctccaaagaaaccgtgggtcaatacgaatcacataccgccttcacgctgccgggcctgtatcgtgtggttcacggtatcgatgtttttgacccgaaattcaatattgtcagtccgggcgcggatatgtccatctattttccgtacaccgaagaaaagcgtcgcctgacgaaattccattcagaaattgaagaactgctgtactcggacgtggaaaacaaggaacacctgtgtgttctgaaagataaaaagaaaccgatcctgtttaccatggcccgtctggatcgcgtgaagaatctgtcaggcctggttgaatggtatggtaaaaacacgcgtctgcgcgaactggcaaatctggtcgtggttggcggtgaccgtcgcaaggaatcgaaagataacgaagaaaaggctgaaatgaagaaaatgtacgatctgatcgaagaatacaagctgaacggccagtttcgttggatcagctctcaaatggaccgtgtgcgcaatggcgaactgtatcgctacatttgcgataccaagggtgcgtttgttcagccggcactgtacgaagctttcggcctgaccgtcgtggaagccatgacgtgcggtctgccgacctttgcgacgtgtaaaggcggtccggccgaaattatcgtgcatggcaaatctggtttccatatcgatccgtatcacggtgatcaggcagctgacaccctggcggatttctttacgaagtgtaaagaagacccgtcacactgggatgaaatttcgaagggcggtctgcaacgtatcgaagaaaaatatacctggcagatttacagccaacgcctgctgaccctgacgggcgtctacggtttttggaaacatgtgtctaatctggatcgcctggaagcccgtcgctatctggaaatgttttacgcactgaagtatcgcccgctggcacaagccgttccgctggcacaggacgactaa

SEQ ID NO: 13 - protein, 1270 aa, Artificial Sequence
  Synthetic polypeptide
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQIAKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSLGSGANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDDWT

SEQ ID NO: 14 - DNA, 3807 bp, Artificial Sequence
  Synthetic polynucleotide
atggagaataagacagaaacaaccgtaagacggaggcggaggattatcttgttccctgtaccatttcagggccatattaatccgatcctccaattagcaaacgtcctctactccaagggattttcaataacaatcttccatactaactttaacaagcctaaaacgagtaattatcctcactttacattcaggttcattctagacaacgaccctcaggatgagcgtatctcaaatttacctacgcatggccccttggcaggtatgcgaataccaataatcaatgagcatggagccgatgaactccgtcgcgagttagagcttctcatgctcgcaagtgaggaagacgaggaagtttcgtgcctaataactgatgcgctttggtacttcgcccaatcagtcgcagactcactgaatctacgccgtttggtccttatgacaagttcattattcaactttcacgcacatgtatcactgccgcaatttgacgagttgggttacctggacccggatgacaaaacgcgattggaggaacaagcgtcgggcttccccatgctgaaagtcaaagatattaagagcgcttatagtaattggcaaattgcgaaagaaattctcggaaaaatgataaagcaaaccaaagcgtcctctggagtaatctggaactccttcaaggagttagaggaatctgaacttgaaacggtcatcagagaaatccccgctccctcgttcttaattccactacccaagcaccttactgcaagtagcagttccctcctagatcatgaccgaaccgtgtttcagtggctggatcagcaacccccgtcgtcagttctatatgtaagctttgggagtacttcggaagtggatgaaaaggacttcttagagattgcgcgagggctcgtggatagcaaacagagcttcctgtgggtagtgagaccgggattcgttaagggctcgacgtgggtcgagccgttgccagatggttttctaggggagagagggagaatcgtgaaatgggttccacagcaagaggttttggctcacggagctataggggccttttggacccactctggttggaattctactcttgaaagtgtctgtgaaggcgttccaatgatattttctgattttgggcttgaccagcctctaaacgctcgctatatgtctgatgtgttgaaggttggcgtgtacctggagaatggttgggaaaggggggaaattgccaacgccatacgccgggtaatggtggacgaggaaggtgagtacatacgtcagaacgctcgggttttaaaacaaaaagcggacgtcagccttatgaagggaggtagctcctatgaatccctagaatccttggtaagctatatatcttcgttaggttctggtgcaaacgctgaacgtatgataacgcgcgtccacagccaacgtgagcgtttgaacgaaacgcttgtttctgagagaaacgaagtccttgccttgctttccagggttgaagccaaaggtaaaggtattttacaacaaaaccagatcattgctgaattcgaagctttgcctgaacaaacccggaagaaacttgaaggtggtcctttctttgaccttctcaaatccactcaggaagcaattgtgttgccaccatgggttgctctagctgtgaggccaaggcctggtgtttgggaatacttacgagtcaatctccatgctcttgtcgttgaagaactccaacctgctgagtttcttcatttcaaggaagaactcgttgatggagttaagaatggtaatttcactcttgagcttgatttcgagccattcaatgcgtctatccctcgtccaacactccacaaatacattggaaatggtgttgacttccttaaccgtcatttatcggctaagctcttccatgacaaggagagtttgcttccattgcttaagttccttcgtcttcacagccaccagggcaagaacctgatgttgagcgagaagattcagaacctcaacactctgcaacacaccttgaggaaagcagaagagtatctagcagagcttaagtccgaaacactgtatgaagagtttgaggccaagtttgaggagattggtcttgagaggggatggggagacaatgcagagcgtgtccttgacatgatacgtcttcttttggaccttcttgaggcgcctgatccttgcactcttgagacttttcttggaagagtaccaatggtgttcaacgttgtgatcctctctccacatggttactttgctcaggacaatgttcttggttaccctgacactggtggacaggttgtttacattcttgatcaagttcgtgctctggagatagagatgcttcaacgtattaagcaacaaggactcaacattaaaccaaggattctcattctaactcgacttctacctgatgcggtaggaactacatgcggtgaacgtctcgagagagtttatgattctgagtactgtgatattcttcgtgtgcccttcagaacagagaagggtattgttcgcaaatggatctcaaggttcgaagtctggccatatctagagacttacaccgaggatgctgcggttgagctatcgaaagaattgaatggcaagcctgaccttatcattggtaactacagtgatggaaatcttgttgcttctttattggctcacaaacttggtgtcactcagtgtaccattgctcatgctcttgagaaaacaaagtacccggattctgatatctactggaagaagcttgacgacaagtaccatttctcatgccagttcactgcggatattttcgcaatgaaccacactgatttcatcatcactagtactttccaagaaattgctggaagcaaagaaactgttgggcagtatgaaagccacacagcctttactcttcccggattgtatcgagttgttcacgggattgatgtgtttgatcccaagttcaacattgtctctcctggtgctgatatgagcatctacttcccttacacagaggagaagcgtagattgactaagttccactctgagatcgaggagctcctctacagcgatgttgagaacaaagagcacttatgtgtgctcaaggacaagaagaagccgattctcttcacaatggctaggcttgatcgtgtcaagaacttgtcaggtcttgttgagtggtacgggaagaacacccgcttgcgtgagctagctaacttggttgttgttggaggagacaggaggaaagagtcaaaggacaatgaagagaaagcagagatgaagaaaatgtatgatctcattgaggaatacaagctaaacggtcagttcaggtggatctcctctcagatggaccgggtaaggaacggtgagctgtaccggtacatctgtgacaccaagggtgcttttgtccaacctgcattatatgaagcctttgggttaactgttgtggaggctatgacttgtggtttaccgactttcgccacttgcaaaggtggtccagctgagatcattgtgcacggtaaatcgggtttccacattgacccttaccatggtgatcaggctgctgatactcttgctgatttcttcaccaagtgtaaggaggatccatctcactgggatgagatctcaaaaggagggcttcagaggattgaggagaaatacacttggcaaatctattcacagaggctcttgacattgactggtgtgtatggattctggaagcatgtctcgaaccttgaccgtcttgaggctcgccgttaccttgaaatgttctatgcattgaagtatcgcccattggctcaggctgttcctcttgcacaagatgattga

SEQ ID NO: 15 - protein, 17 aa, Artificial Sequence
  Synthetic polypeptide
YKDDSGYSSSYAAAAGM

(19) United States
(12) Patent Application Publication
(10) Pub. No.: US 2022/0205007 A1
(43) Pub. Date: Jun. 30, 2022

(54) BIOSYNTHETIC PRODUCTION OF STEVIOL GLYCOSIDE REBAUDIOSIDE I VIA VARIANT ENZYMES

(71) Applicant: Conagen Inc., Bedford, MA (US)

(72) Inventors: Guohong Mao, Burlington, MA (US); Michael Batten, Westford, MA (US); Xiaodan Yu, Lexington, MA (US)

(73) Assignee: Conagen Inc., Bedford, MA (US)

(21) Appl. No.: 17/570,544

(22) Filed: Jan. 7, 2022

Related U.S. Application Data
(63) Continuation of application No. PCT/US2020/041394, filed on Jul. 9, 2020.
Application No. PCT/US2020/041394 is a continuation-in-part of application No. 16/679,032, filed on Nov. 8, 2019, now Pat. No. 10,883,130.
Application No. 16/679,032 is a continuation of application No. PCT/US2019/021876, filed on Mar. 12, 2019.
Application No. PCT/US2020/041394 is a continuation-in-part of application No. 16/506,892, filed on Jul. 9, 2019.
Application No. 16/506,892 is a continuation-in-part of application No. PCT/US2019/021876, filed on Mar. 12, 2019.
(60) Provisional application No. 62/695,252, filed on Jul. 9, 2018.
(60) Provisional application No. 62/682,260, filed on Jun. 8, 2018.
(60) Provisional application No. 62/641,590, filed on Mar. 12, 2018.
(60) Provisional application No. 62/695,252, filed on Jul. 9, 2018.

(51) Int. Cl. C12P 19/18; C12N 9/10

(52) U.S. Cl. CPC C12P 19/18; C12Y 204/01013; C12N 9/1062; C12N 9/1051

(57) ABSTRACT

The present invention relates, at least in part, to the production of steviol glycoside rebaudioside I through the use of variant UGT enzymes having activity to transfer a glucosyl group from UDP-glucose to rebaudioside A to produce rebaudioside I.

### RELATED APPLICATIONS

This application is a continuation of International Patent Application No. PCT/US2020/041394, filed Jul. 9, 2020, entitled “BIOSYNTHETIC PRODUCTION OF STEVIOL GLYCOSIDE REBAUDIOSIDE I VIA VARIANT ENZYMES”, which is a continuation in part of U.S. patent application Ser. No. 16/506,892, filed Jul. 9, 2019 and entitled “BIOSYNTHETIC PRODUCTION OF STEVIOL GLYCOSIDE REBAUDIOSIDE I VIA VARIANT ENZYMES,” and is a continuation in part of U.S. patent application Ser. No. 16/679,032, filed Nov. 8, 2019 and entitled “BIOSYNTHETIC PRODUCTION OF STVIOL GLYCOSIDES REBAUDIOSIDE J AND REBAUDIOSIDE N”, the entire contents of each of which are incorporated herein by reference. U.S. patent application Ser. No. 16/506,892 claims priority to U.S. Provisional Application 62/695,252, filed Jul. 9, 2018 and entitled “BIOSYNTHETIC PRODUCTION OF STEVIOL GLYCOSIDE REBAUDIOSIDE I VIA VARIANT ENZYMES,” and is a continuation in part of International Patent Application No. PCT/US2019/021876, filed Mar. 12, 2019 and entitled “BIOSYNTHETIC PRODUCTION OF STEVIOL GLYCOSIDES REBAUDIOSIDE J AND REBAUDIOSIDE N,” which claims priority to U.S. Provisional Application 62/695,252, filed Jul. 9, 2018 and entitled “BIOSYNTHETIC PRODUCTION OF STEVIOL GLYCOSIDE REBAUDIOSIDE I VIA VARIANT ENZYMES,” U.S. Provisional Application No. 62/682,260, filed Jun. 8, 2018, and entitled “BIOSYNTHETIC PRODUCTION OF STEVIOL GLYCOSIDES REBAUDIOSIDE J AND REBAUDIOSIDE N” and U.S. Provisional 62/641,590, filed Mar. 12, 2018, and entitled “BIOSYNTHETIC PRODUCTION OF STEVIOL GLYCOSIDES REBAUDIOSIDE J AND REBAUDIOSIDE N”, the entire contents of each of which are incorporated herein by reference.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Jan. 6, 2022, is named C149770032US01-SEQ-ZJG and is 67,040 bytes in size.

### FIELD OF THE INVENTION

The field of the invention relates, at least in part, to methods and processes useful in the production of a specific steviol glycoside via a biosynthetic pathway engineered into selected microorganisms. More specifically, the present disclosure provides for the production of Rebaudioside I (“Reb I”) using previously unknown enzymes and/or enzyme variants.

### SUMMARY OF THE INVENTION

The present invention is focused, at least in part, on the production of Reb I from Reb A and/or the production of Reb I through the use of modified enzymes.

The specific and directed glycosylation of rebaudioside A (at the C-19-O-glucose) can produce rebaudioside Reb I. The synthetic steps to produce Reb I from Reb A enzymatically have been accomplished herein with alternative enzymes. As described in more detail below, it has been found that mutations in the domains in UGT76G1 can cause specific alterations of glucosylation activity.

In addition, methods of producing rebaudioside I from stevioside through rebaudioside A at high titer and/or with a reduction in cost are provided.

The present invention encompasses a method of producing Reb I from Reb A. In particular, the current invention provides for the production of steviol glycoside rebaudioside I “Reb I” which is identified as (13-[(2-O-β-D-glucopyranosyl-3-O-β-D-glucopyranosyl-β-D-glucopyranosyl)oxy] ent-kaur-16-en-19-oic acid-[(3-O-β-D-glucopyranosyl-β-D-glucopyranosyl) ester].

Provided herein, inter alia, are methods for synthesizing rebaudioside I, the method comprising preparing a reaction mixture comprising: (a) a steviol glycoside composition comprising rebaudioside A; (b) a substrate selected from the group consisting of sucrose, uridine diphosphate (UDP), uridine diphosphate-glucose (UDP-glucose), and combinations thereof; and (c) a UDP-glycosyltransferase enzyme comprising the amino acid sequence of SEQ ID NO: 1; and incubating the reaction mixture for a sufficient time to produce rebaudioside I.

In some embodiments of any one of the methods provided, the UDP-glycosyltransferase enzyme used in the methods described herein comprises an amino acid sequence that is at least 80% (e.g., at least 80%, at least 85%, at least 90%, at least 95%, at least 96%, at least 97%, at least 98%, at least 99% or 100%) identical to SEQ ID NO: 1.

In one aspect, any one of the enzymes described herein is provided.

In some embodiments of any one of the methods provided, the steviol glycoside composition is stevia extract.

In some embodiments of any one of the methods provided, the methods described herein further comprises adding a sucrose synthase to the reaction mixture. In some embodiments of any one of the methods provided, the sucrose synthase is an Arabidopsis thaliana sucrose synthase 1 (AtSUS1) comprising the amino acid sequence of SEQ ID NO: 11.

In some embodiments of any one of the methods provided, the sucrose synthase used in the methods described herein comprises an amino acid sequence that is at least 80% (e.g., at least 80%, at least 85%, at least 90%, at least 95%, at least 96%, at least 97%, at least 98%, at least 99% or 100%) identical to SEQ ID NO: 11.

In some embodiments of any one of the methods provided, the UDP-glycosyltransferase enzyme used is an UGT76G1 L200A mutant (LA) -AtSUS1 fusion enzyme. In some embodiments, the LA-AtSUS1 fusion enzyme used in the methods described herein comprises an amino acid sequence that is at least 80% (e.g., at least 80%, at least 85%, at least 90%, at least 95%, at least 96%, at least 97%, at least 98%, at least 99% or 100%) identical to SEQ ID NO: 13. In some embodiments, the LA-AtSUS1 fusion enzyme used in the methods described herein comprises the amino acid sequence of SEQ ID NO: 13.

In some embodiments of any one of the methods provided, the reaction mixture is in vitro, i.e., the method described herein is performed in vitro. For in vitro reactions, the UDP-glycosyltransferase enzyme and/or the sucrose synthase can be added to the in vitro reaction mixture.

In some embodiments of any one of the methods provided, the reaction mixture is a cell-based reaction mixture, i.e., the reaction is performed in a cell. For cell-based reactions, the UDP-glycosyltransferase enzyme and/or the sucrose synthase can be expressed in a host cell.

In some embodiments of any one of the methods provided, the UDP-glycosyltransferase enzyme and/or the sucrose synthase are expressed from nucleotide sequences encoding UDP-glycosyltransferase enzyme and/or the sucrose synthase, respectively. As such, in some embodiments of any one of the methods provided, the host cell comprises a nucleotide sequence having at least 80% (e.g., at least 80%, at least 85%, at least 90%, at least 95%, at least 96%, at least 97%, at least 98%, at least 99% or 100%) identity to SEQ ID NO: 2. In some embodiments of any one of the methods provided, the host cell further comprises a nucleotide sequence having at least 80% (e.g., at least 80%, at least 85%, at least 90%, at least 95%, at least 96%, at least 97%, at least 98%, at least 99% or 100%) identity to SEQ ID NO: 12. In some embodiments of any one of the methods provided, the host cell further comprises a nucleotide sequence having at least 80% (e.g., at least 80%, at least 85%, at least 90%, at least 95%, at least 96%, at least 97%, at least 98%, at least 99% or 100%) identity to SEQ ID NO: 14.

In one aspect, a nucleic acid comprising any one of the sequences described herein is provided.

In some embodiments of any one of the methods provided, the host cell is selected from the group consisting of a yeast, a non-steviol glycoside producing plant, an alga, a fungus, and a bacterium.

In some embodiments of any one of the methods provided, the host cell is selected from the group consisting of Escherichia; Salmonella; Bacillus; Acinetobacter; Streptomyces; Corynebacterium; Methylosinus; Methylomonas; Rhodococcus; Pseudomonas; Rhodobacter; Synechocystis; Saccharomyces; Zygosaccharomyces; Kluyveromyces; Candida; Hansenula; Debaryomyces; Mucor; Pichia; Torulopsis; Aspergillus; Arthrobotlys; Brevibacteria; Microbacterium; Arthrobacter; Citrobacter; Klebsiella; Pantoea; and Clostridium.

In some embodiments of any one of the methods provided, the host cell is a cell isolated from plants selected from the group consisting of soybean; rapeseed; sunflower; cotton; corn; tobacco; alfalfa; wheat; barley; oats; sorghum; rice; broccoli; cauliflower; cabbage; parsnips; melons; carrots; celery; parsley; tomatoes; potatoes; strawberries; peanuts; grapes; grass seed crops; sugar beets; sugar cane; beans; peas; rye; flax; hardwood trees; softwood trees; forage grasses; Arabidopsis thaliana; rice (Oryza sativa); Hordeum yulgare; switchgrass (Panicum vigratum); Brachypodium spp.; Brassica spp.; and Crambe abyssinica.

In some embodiments of any one of the methods provided, the host cell is a bacterial cell (e.g., an E. coli cell).

In some embodiments of any one of the methods provided, the host cell is a yeast cell (e.g., a Saccharomyces cerevisiae cell).

In one aspect, any one of the host cells described herein is provided.

In some embodiments of any one of the methods provided, the subtrate is UDP-glucose. In some embodiments of any one of the methods provided, the UDP-glycose is generated in situ (e.g., from UDP and sucrose using a sucrose synthase).

In some embodiments of any one of the methods provided, the rebaudioside A has a concentration of 15 to 50 g/L (e.g., 15-50, 20-50, 30-50, 40-50, 15-40, 20-40, 30-40, 30-50, 30-40, or 40-50 g/L) in the reaction mixture.

In some embodiments of any one of the methods provided, the reaction mixture has a pH range of 6.5 to 9.5 (e.g., 6.5, 7, 7.5, 8, 8.5, 9, or 9.5) at a temperature of 35° C. to 45 ° C. (e.g., 35° C., 36° C., 37° C., 38° C., 39° C., 40° C., 41° C., 42° C., 43° C., 44° C., or 45° C.).

In some embodiments of any one of the methods provided, the method further comprises isolating crude rebaudioside I (e.g., using a microporous adsorption resin).

In one aspect, a composition comprising any one of the rebaudioside I compositions described herein is provided.

In some embodiments of any one of the methods provided, the method described herein further comprises crystallizing the crude rebaudioside Ito obtain rebaudioside I with a purity of greater than 98% (e.g., 98%, 99%, or 99.9%).

Aspects of the present disclosure provide a mutant of the UGT76G1 enzyme comprising a L200A mutation (herein termed the “LA mutant”). The L200A mutation is relative to SEQ ID NO: 9. In some embodiments of any one of the compositions or methods provided, the LA mutant comprises an amino acid sequence having at least 80% (e.g., at least 80%, at least 85%, at least 90%, at least 95%, at least 96%, at least 97%, at least 98%, at least 99% or 100%) identity to SEQ ID NO: 1, and comprises the L200A mutation. In some embodiments of any one of the compositions or methods provided, the LA mutant comprises the amino acid sequence of SEQ ID NO: 1.

Aspects of the present disclosure provide fusion protein of the LA mutant fused to AtSUS1. In some embodiments of any one of the compositions or methods provided, the LA-AtSUS1 fusion protein comprises an amino acid sequence having at least 80% (e.g., at least 80%, at least 85%, at least 90%, at least 95%, at least 96%, at least 97%, at least 98%, at least 99% or 100%) identity to SEQ ID NO: 13. In some embodiments of any one of the compositions or methods provided, the LA-AtSUS1 fusion protein comprises the amino acid sequence of SEQ ID NO: 13.

In one aspect, a composition comprising any one of the mutants or fusion proteins described herein is provided.

In terms of product/commercial utility there are several dozen products containing steviol glycosides on the market in the United States and can be used in everything from analgesics to pest repellents as well as in foods and as a dietary supplement. Products containing steviol glycosides can be aerosols, liquids, gels or granular formulations and such products are provided in some embodiments.

While the disclosure is susceptible to various modifications and alternative forms, specific embodiments thereof are shown by way of example in the drawing and will herein be described in detail. It should be understood, however, that the drawings and detailed description presented herein are not intended to limit the disclosure to the particular embodiment disclosed, but on the contrary, the intention is to cover all modifications, equivalents, and alternatives falling within the spirit and scope of the present disclosure as defined by the appended claims.

Other features and advantages of this invention will become apparent in the following detailed description of embodiments of this invention.

### BRIEF DESCRIPTION OF THE DRAWINGS

FIG. 1 shows the chemical structure of rebaudioside I (“Reb I”), (C₅₀H₈₀O₂₈), 13-[(2-O-β-D-glucopyranosyl-3-O-β-D-glucopyranosyl)-β-D-glucopyranosyl)oxy]ent-kaur-16-en-19oic acid-(3-O-β-D-glucopyranosyl)-β-D-glucopyranosyl), ester.

FIG. 2 shows the biosynthesis pathway of Reb I from Reb A.

FIGS. 3A-3F show the in vitro production of Reb I from Reb A catalyzed by selected UGTs after 6 hours of incubation. FIG. 3A shows the standards of rebaudioside A (Reb A) and rabaudioside I (Reb I). FIGS. 3B-3F, respectively, show the amount of Reb I enzymatically produced from Reb A by UGT76G1 (FIG. 3B), CP1 (FIG. 3C), CP2 (FIG. 3D), LA (FIG. 3E) and UGT76G1-AtSUS1 fusion enzyme (GS) (FIG. 3F).

FIGS. 4A-4D show the in vitro production of Reb I from Reb A catalyzed by UGT76G1, a coupling system in which both UGT76G1 and AtSUS1 are present, and a UGT76G1-AtSUS1 fusion enzyme (“GS”), with the addition of UDP and sucrose. FIG. 4A shows the standards of rebaudioside I (Reb I) and rebaudioside A (Reb A). FIGS. 4B-4D, respectively, show the amount of Reb I converted from Reb A in an enzymatic reaction catalyzed by UGT76G1 (FIG. 4B), a coupling system in which both UGT76G1 and AtSUS1 are present (FIG. 4C), and a UGT76G1-AtSUS1 fusion enzyme (GS) (FIG. 4D).

FIG. 5 shows the key TOCSY and HMBC correlations of rebaudioside I.

### DETAILED DESCRIPTION OF CERTAIN EMBODIMENTS

Steviol Glycosides are a class of chemical compounds responsible for the sweet taste of the leaves of the South American plant Stevia rebaudiana (Asteraceae), and can be used as sweeteners in food, feed and beverages.

### Definitions:

Cellular system includes any cell that provides for the expression of ectopic proteins. It includes bacteria, yeast, plant cells and animal cells or any cellular system that would allow the genetic transformation with the selected genes and thereafter the biosynthetic production of the desired steviol glycosides from steviol. It includes both prokaryotic and eukaryotic cells. It also includes the in vitro expression of proteins based on cellular components, such as ribosomes. E. coli is a preferred microbial system in an embodiment of any one of the methods provided herein.

Coding sequence is to be given its ordinary and customary meaning to a person of ordinary skill in the art and is used without limitation to refer to a DNA sequence that encodes for a specific amino acid sequence.

Growing the Cellular System. Growing includes providing an appropriate medium that would allow cells to multiply and divide. It also includes providing resources so that cells or cellular components can translate and make recombinant proteins.

Protein Expression. Protein production can occur after gene expression. It consists of the stages after DNA has been transcribed to messenger RNA (mRNA). The mRNA is then translated into polypeptide chains, which are ultimately folded into proteins. DNA is present in the cells through transfection—a process of deliberately introducing nucleic acids into cells. The term is often used for non-viral methods in eukaryotic cells. It may also refer to other methods and cell types, although other terms are preferred: “transformation” is more often used to describe non-viral DNA transfer in bacteria, non-animal eukaryotic cells, including plant cells. In animal cells, transfection is the preferred term as transformation is also used to refer to progression to a cancerous state (carcinogenesis) in these cells. Transduction is often used to describe virus-mediated DNA transfer. Transformation, transduction, and viral infection are included under the definition of transfection for this application.

Yeast. According to the current invention yeast as claimed herein are eukaryotic, single-celled microorganisms classified as members of the fungus kingdom. Yeasts are unicellular organisms which evolved from multicellular ancestors but with some species useful for the current invention being those that have the ability to develop multicellular characteristics by forming strings of connected budding cells known as pseudohyphae or false hyphae.

The names of the UGT enzymes used in the present disclosure are consistent with the nomenclature system adopted by the UGT Nomenclature Committee (Mackenzie et al., “The UDP glycosyltransferase gene super family: recommended nomenclature updated based on evolutionary divergence,” PHARMACOGENETICS, 1997, vol. 7, pp. 255-269), which classifies the UGT genes by the combination of a family number, a letter denoting a subfamily, and a number for an individual gene. For example, the name “UGT76G1” refers to a UGT enzyme encoded by a gene belonging to UGT family number 76 (which is of plant origin), subfamily G, and gene number 1.

### Structural Terms:

As used herein, the singular forms “a, an” and “the” include plural references unless the content clearly dictates otherwise.

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

The term “complementary” is to be given its ordinary and customary meaning to a person of ordinary skill in the art and is used without limitation to describe the relationship between nucleotide bases that are capable of hybridizing to one another. For example, with respect to DNA, adenosine is complementary to thymine and cytosine is complementary to guanine. Accordingly, the subjection technology also includes isolated nucleic acid fragments that are complementary to the complete sequences as reported in the accompanying Sequence Listing as well as those substantially similar nucleic acid sequences

The terms “nucleic acid” and “nucleotide” are to be given their respective ordinary and customary meanings to a person of ordinary skill in the art, and are used without limitation to refer to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally-occurring nucleotides. Unless otherwise indicated, a particular nucleic acid sequence also implicitly encompasses conservatively modified or degenerate variants thereof (e.g., degenerate codon substitutions) and complementary sequences, as well as the sequence explicitly indicated.

The term “isolated” is to be given its ordinary and customary meaning to a person of ordinary skill in the art, and when used in the context of an isolated nucleic acid or an isolated polypeptide, is used without limitation to refer to a nucleic acid or polypeptide that, by the hand of man, exists apart from its native environment and is therefore not a product of nature. An isolated nucleic acid or polypeptide can exist in a purified form or can exist in a non-native environment such as, for example, in a transgenic host cell.

The terms “incubating” and “incubation” as used herein means a process of mixing two or more chemical or biological entities (such as a chemical compound and an enzyme) and allowing them to interact under conditions favorable for producing a steviol glycoside composition.

The term “degenerate variant” refers to a nucleic acid sequence having a residue sequence that differs from a reference nucleic acid sequence by one or more degenerate codon substitutions. Degenerate codon substitutions can be achieved by generating sequences in which the third position of one or more selected (or all) codons is substituted with mixed base and/or deoxy inosine residues. A nucleic acid sequence and all of its degenerate variants will express the same amino acid or polypeptide.

The terms “polypeptide,” “protein,” and “peptide” are to be given their respective ordinary’ and customary meanings to a person of ordinary skill in the art; the three terms are sometimes used interchangeably, and are used without limitation to refer to a polymer of amino acids, or amino acid analogs, regardless of its size or function. Although “protein” is often used in reference to relatively large polypeptides, and “peptide” is often used in reference to small polypeptides, usage of these terms in the art overlaps and varies. The term ‘polypeptide” as used herein refers to peptides, polypeptides, and proteins, unless otherwise noted. The terms “protein,” “polypeptide,” and “peptide” are used interchangeably herein when referring to a polynucleotide product. Thus, exemplary polypeptides include polynucleotide products, naturally occurring proteins, homologs, orthologs, paralogs, fragments and other equivalents, variants, and analogs of the foregoing.

The terms “polypeptide fragment” and “fragment,” when used in reference to a reference polypeptide, are to be given their ordinary and customary meanings to a person of ordinary skill in the art, and are used without limitation to refer to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both.

The term “functional fragment” of a polypeptide or protein refers to a peptide fragment that is a portion of the full-length polypeptide or protein, and has substantially the same biological activity, or carries out substantially the same function as the full-length polypeptide or protein (e.g., carrying out the same enzymatic reaction).

The terms “variant polypeptide,” “modified amino acid sequence” or “modified polypeptide,” which are used interchangeably, refer to an amino acid sequence that is different from the reference polypeptide by one or more amino acids, e.g., by one or more amino acid substitutions, deletions, and/or additions. In an aspect, a variant is a “functional variant” which retains some or all of the ability of the reference polypeptide.

The term “functional variant” further includes conservatively substituted variants. The term “conservatively substituted variant” refers to a peptide having an amino acid sequence that differs from a reference peptide by one or more conservative amino acid substitutions and maintains some or all of the activity of the reference peptide. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another such as between arginine and lysine, between glutamine and asparagine, between threonine and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. Such substitutions are expected to have little or no effect on the apparent molecular weight or isoelectric point of the protein or polypeptide. The phrase “conservatively substituted variant” also includes peptides wherein a residue is replaced with a chemically-derivatized residue, provided that the resulting peptide maintains some or all of the activity of the reference peptide as described herein.

The term “variant,” in connection with the polypeptides of the subject technology, further includes a functionally active polypeptide having an amino acid sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical to the amino acid sequence of a reference polypeptide.

The term “homologous” in all its grammatical forms and spelling variations refers to the relationship between polynucleotides or polypeptides that possess a “common evolutionary origin,” including polynucleotides or polypeptides from super families and homologous polynucleotides or proteins from different species (Reeck et al., CELL 50:667, 1987). Such polynucleotides or polypeptides have sequence homology, as reflected by their sequence similarity, whether in terms of percent identity or the presence of specific amino acids or motifs at conserved positions. For example, two homologous polypeptides can have amino acid sequences that are at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 900 at least 91%, at least 92%, at least 93%, at least 94%, at least 95%, at least 96%, at least 97%, at least 98%, at least 99%, and even 100% identical.

“Suitable regulatory sequences” is to be given its ordinary and customary meaning to a person of ordinary skill in the art, and is used without limitation to refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” is to be given its ordinary and customary meaning to a person of ordinary skill in the art and is used without limitation to refer to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters, which cause a gene to be expressed in most cell types at most times, are commonly referred to as “constitutive promoters.” It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it can affect the expression of that coding sequence (i.e., that the coding sequence is under the transcriptional control of the promoter). Coding sequences can be operably linked to regulatory sequences in sense or antisense orientation.

The term “expression” as used herein, is to be given its ordinary and customary meaning to a person of ordinary skill in the art, and is used without limitation to refer to the transcription and stable accumulation of sense (mRNA) or antisense RNA derived from the nucleic acid fragment of the subject technology. “Over-expression” refers to the production of a gene product in transgenic or recombinant organisms that exceeds levels of production in normal or non-transformed organisms.

“Transformation” is to be given its ordinary and customary meaning to a person of reasonable skill in the craft, and is used without limitation to refer to the transfer of a polynucleotide into a target cell. The transferred polynucleotide can be incorporated into the genome or chromosomal DNA of a target cell, resulting in genetically stable inheritance, or it can replicate independent of the host chromosomal. Host organisms containing the transformed nucleic acid fragments are referred to as “transgenic” or “transformed”.

The terms “transformed,” “transgenic,” and “recombinant,” when used herein in connection with host cells, are to be given their respective ordinary and customary meanings to a person of ordinary skill in the art and are used without limitation to refer to a cell of a host organism, such as a plant or microbial cell, into which a heterologous nucleic acid molecule has been introduced. The nucleic acid molecule can be stably integrated into the genome of the host cell, or the nucleic acid molecule can be present as an extrachromosomal molecule. Such an extrachromosomal molecule can be auto-replicating. Transformed cells, tissues, or subjects are understood to encompass not only the end product of a transformation process, but also transgenic progeny thereof.

The terms “recombinant,” “heterologous,” and “exogenous,” when used herein in connection with polynucleotides, are to be given their ordinary and customary meanings to a person of ordinary skill in the art and are used without limitation to refer to a polynucleotide (e.g., a DNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found.

Similarly, the terms “recombinant,” “heterologous,” and “exogenous,” when used herein in connection with a polypeptide or amino acid sequence, means a polypeptide or amino acid sequence that originates from a source foreign to the particular host cell or, if from the same source, is modified from its original form. Thus, recombinant DNA segments can be expressed in a host cell to produce a recombinant polypeptide.

The terms “plasmid,” “vector,” and “cassette” are to be given their respective ordinary and customary meanings to a person of ordinary skill in the art and are used without limitation to refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

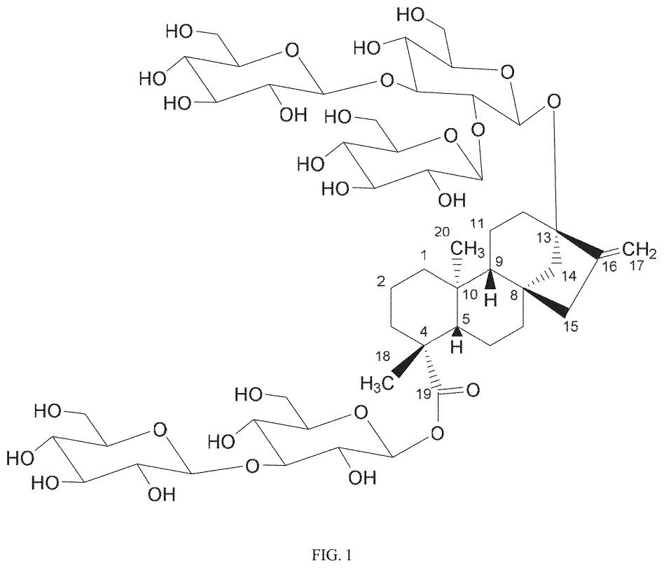

The present invention relates to the production of a steviol glycoside of interest, Reb I from using UGT enzymes to allow that conversion. The subject technology provides recombinant polypeptides with UDP glycosyltransferase activities, such as 1,3-19-O-glucose glycosylation activity and 1,3-13-O-glucose glycosylation activity for synthesizing steviol glycosides. The recombinant polypeptide of the subject technology is useful for the biosynthesis of steviol glycoside compounds. In the present disclosure, UDP-glycosyltransferase (UGT) refers to an enzyme that transfers a sugar residue from an activated donor molecule (typically UDP-glucose) to an acceptor molecule. The 1,3-19-O-glucose glycosylation activity refers to an enzymatic activity that transfers a sugar moiety to the C-3′ of the 19-O glucose moiety of rebaudioside A to produce rebaudioside I (Reb I) (FIG. 1).

### Synthetic Biology

Standard recombinant DNA and molecular cloning techniques used here are well known in the art and are described, for example, by Sambrook, J., Fritsch, E. F. and Maniatis, T. MOLECULAR CLONING: A LABORATORY MANUAL, 2nd ed.; Cold Spring Harbor Laboratory: Cold Spring Harbor, N.Y., 1989 (hereinafter “Maniatis”); and by Silhavy, T. J., Bennan, M. L. and Enquist, L. W. EXPERIMENTS WITH GENE FUSIONS; Cold Spring Harbor Laboratory: Cold Spring Harbor, N.Y., 1984; and by Ausubel, F. M. et al., IN CURRENT PROTOCOLS IN MOLECULAR BIOLOGY, published by Greene Publishing and Wiley-Interscience, 1987; (the entirety of each of which is hereby incorporated herein by reference).

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred materials and methods are described below.

The disclosure will be more fully understood upon consideration of the following non-limiting Examples. It should be understood that these Examples, while indicating preferred embodiments of the subject technology, are given by way of illustration only. From the above discussion and these Examples, one skilled in the art can ascertain the essential characteristics of the subject technology, and without departing from the spirit and scope thereof, can make various changes and modifications of the subject technology to adapt it to various uses and conditions.

Glycosylation is often considered a ubiquitous reaction controlling the bioactivity and storage of plant natural products. Glycosylation of small molecules is catalyzed by a superfamily of transferases in most plant species that have been studied to date. These glycosyltransferases (GTs) have been classified into over 60 families. Of these, the family 1 GT enzymes, also known as the UDP glycosyltransferases (UGTs), transfer UDP-activated sugar moieties to specific acceptor molecules. These are the molecules that transfer such sugar moieties in the steviol glycosides to create various rebaudiosides. Each of these UGTs have their own activity profile and preferred structure locations where they transfer their activated sugar moieties.

### Production Systems

Expression of proteins in prokaryotes is most often carried out in a bacterial host cell with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such vectors are within the scope of the present disclosure.

In an embodiment, the expression vector includes those genetic elements for expression of the recombinant polypeptide in bacterial cells. The elements for transcription and translation in the bacterial cell can include a promoter, a coding region for the protein complex, and a transcriptional terminator.

A person of ordinary skill in the art will be aware of the molecular biology techniques available for the preparation of expression vectors. The polynucleotide used for incorporation into the expression vector of the subject technology, as described above, can be prepared by routine techniques such as polymerase chain reaction (PCR).

Several molecular biology techniques have been developed to operably link DNA to vectors via complementary cohesive termini. In one embodiment, complementary homopolymer tracts can be added to the nucleic acid molecule to be inserted into the vector DNA. The vector and nucleic acid molecule are then joined by hydrogen bonding between the complementary homopolymeric tails to form recombinant DNA molecules.

In an alternative embodiment, synthetic linkers containing one or more restriction sites provide are used to operably link the polynucleotide of the subject technology to the expression vector. In an embodiment, the polynucleotide is generated by restriction endonuclease digestion. In an embodiment, the nucleic acid molecule is treated with bacteriophage T4 DNA polymerase or E. coli DNA polymerase I, enzymes that remove protruding, 3′-single-stranded termini with their 3′-5′-exonucleolytic activities, and fill in recessed 3′-ends with their polymerizing activities, thereby generating blunt ended DNA segments. The blunt-ended segments are then incubated with a large molar excess of linker molecules in the presence of an enzyme that can catalyze the ligation of blunt-ended DNA molecules, such as bacteriophage T4 DNA ligase. Thus, the product of the reaction is a polynucleotide carrying polymeric linker sequences at its ends. These polynucleotides are then cleaved with the appropriate restriction enzyme and ligated to an expression vector that has been cleaved with an enzyme that produces termini compatible with those of the polynucleotide.

Alternatively, a vector having ligation-independent cloning (LIC) sites can be employed. The required PCR amplified polynucleotide can then be cloned into the LIC vector without restriction digest or ligation (Aslanidis and de Jong, NUCL. ACID. RES. 18 6069-74, (1990), Haun, et al, BIOTECHNIQUES 13, 515-18 (1992), which is incorporated herein by reference to the extent it is consistent herewith).

In an embodiment, to isolate and/or modify the polynucleotide of interest for insertion into the chosen plasmid, it is suitable to use PCR. Appropriate primers for use in PCR preparation of the sequence can be designed to isolate the required coding region of the nucleic acid molecule, add restriction endonuclease or LIC sites, place the coding region in the desired reading frame.

In an embodiment, a polynucleotide for incorporation into an expression vector of the subject technology is prepared using PCR using appropriate oligonucleotide primers. The coding region is amplified, whilst the primers themselves become incorporated into the amplified sequence product. In an embodiment, the amplification primers contain restriction endonuclease recognition sites, which allow the amplified sequence product to be cloned into an appropriate vector.

The expression vectors can be introduced into plant or microbial host cells by conventional transformation or transfection techniques. Transformation of appropriate cells with an expression vector of the subject technology is accomplished by methods known in the art and typically depends on both the type of vector and cell. Suitable techniques include calcium phosphate or calcium chloride co-precipitation, DEAE-dextran mediated transfection, lipofection, chemoporation or electroporation.

Successfully transformed cells, that is, those cells containing the expression vector, can be identified by techniques well known in the art. For example, cells transfected with an expression vector of the subject technology can be cultured to produce polypeptides described herein. Cells can be examined for the presence of the expression vector DNA by techniques well known in the art.

The host cells can contain a single copy of the expression vector described previously, or alternatively, multiple copies of the expression vector,

In some embodiments, the transformed cell is an animal cell, an insect cell, a plant cell, an algal cell, a fungal cell, or a yeast cell. In some embodiments, the cell is a plant cell selected from the group consisting of: canola plant cell, a rapeseed plant cell, a palm plant cell, a sunflower plant cell, a cotton plant cell, a corn plant cell, a peanut plant cell, a flax plant cell, a sesame plant cell, a soybean plant cell, and a petunia plant cell.

Microbial host cell expression systems and expression vectors containing regulatory sequences that direct highlevel expression of foreign proteins are well known to those skilled in the art. Any of these could be used to construct vectors for expression of the recombinant polypeptide of the subjection technology in a microbial host cell. These vectors could then be introduced into appropriate microorganisms via transformation to allow for high level expression of the recombinant polypeptide of the subject technology.

Vectors or cassettes useful for the transformation of suitable microbial host cells are well known in the art. Typically the vector or cassette contains sequences directing transcription and translation of the relevant polynucleotide, a selectable marker, and sequences allowing autonomous replication or chromosomal integration. Suitable vectors comprise a region 5′ of the polynucleotide which harbors transcriptional initiation controls and a region 3′ of the DNA fragment which controls transcriptional termination. It is preferred for both control regions to be derived from genes homologous to the transformed host cell, although it is to be understood that such control regions need not be derived from the genes native to the specific species chosen as a host.

Initiation control regions or promoters, which are useful to drive expression of the recombinant polypeptide in the desired microbial host cell are numerous and familiar to those skilled in the art. Virtually any promoter capable of driving these genes is suitable for the subject technology including but not limited to CYCI, HIS3, GALI, GALIO, ADHI, PGK, PH05, GAPDH, ADCI, TRPI, URA3, LEU2, ENO, TPI (useful for expression in Saccharomyces); AOXI (useful for expression in Pichia); and lac, trp, JPL, IPR, T7, tac, and trc (useful for expression in Escherichia coli).

Termination control regions may also be derived from various genes native to the microbial hosts. A termination site optionally may be included for the microbial hosts described herein.

In plant cells, the expression vectors of the subject technology can include a coding region operably linked to promoters capable of directing expression of the recombinant polypeptide of the subject technology in the desired tissues at the desired stage of development. For reasons of convenience, the polynucleotides to be expressed may comprise promoter sequences and translation leader sequences derived from the same polynucleotide. 3′ non-coding sequences encoding transcription termination signals should also be present. The expression vectors may also comprise one or more introns to facilitate polynucleotide expression.

For plant host cells, any combination of any promoter and any terminator capable of inducing expression of a coding region may be used in the vector sequences of the subject technology. Some suitable examples of promoters and terminators include those from nopaline synthase (nos), octopine synthase (ocs) and cauliflower mosaic virus (CaMV) genes. One type of efficient plant promoter that may be used is a high-level plant promoter. Such promoters, in operable linkage with an expression vector of the subject technology should be capable of promoting the expression of the vector. High level plant promoters that may be used in the subject technology include the promoter of the small subunit (ss) of the ribulose-1, 5-bisphosphate carboxylase for example from soybean (Berry-Lowe et al., J. MOLECULAR AND APP. GEN., 1:483 498 (1982), the entirety of which is hereby incorporated herein to the extent it is consistent herewith), and the promoter of the chlorophyll alb binding protein. These two promoters are known to be light-induced in plant cells (see, for example, GENETIC ENGINEERING OF PLANTS, AN AGRICULTURAL PERSPECTIVE, A. Cashmore, Plenum, N.Y. (1983), pages 29 38; Coruzzi, G. et al., The Journal of Biological CHEMISTRY, 258: 1399 (1983), and Dunsmuir, P. et al., JOURNAL OF MOLECULAR AND APPLIED GENETICS, 2:285 (1983), each of which is hereby incorporated herein by reference to the extent they are consistent herewith).

### Precursor Synthesis to Reb I

As previously stated steviol glycosides are the chemical compounds responsible for the sweet taste of the leaves of the South American plant Stevia rebaudiana (Asteraceae) and in the plant Rubus chingii (Rosaceae). These compounds are glycosylated diterpenes. Specifically, their molecules can be viewed as a steviol molecule, with its hydroxyl hydrogen atom replaced by a glucose molecule to form an ester, and a hydroxyl hydrogen with combinations of glucose and rhamnose to form an acetal.

One method of making the compounds of interest in the current invention is to take common or inexpensive precursors such as steviol or rubososide derived chemically or produced via biosynthesis in engineered microbes such as bacteria and/or yeast and to synthesize targeted steviol glycosides through known or inexpensive methods, such as Reb I.

Aspects of the present invention relate to methods involving recombinantly expressing enzymes in a microbial system capable of producing steviol. In general, such enzymes may include: a copalyl diphosphate synthase (CPS), a kaurene synthase (KS) and a geranylgeranyl diphosphate to synthase (GGPPS) enzyme. This should occur in a microbial strain that expresses an endogenous isoprenoid synthesis pathway, such as the non-mevalonate (MEP) pathway or the mevalonic acid pathway (MVA). In some embodiments, the cell is a bacterial cell, including E. coli, or yeast cell such as a Saccharomyces cell, Pichia cell, or a Yarrowia cell. In some embodiments, the cell is an algal cell or a plant cell.

Thereafter, the precursor is recovered from the fermentation culture for use in chemical synthesis. Typically, this is steviol though it can be kaurene, or a steviol glycoside from the cell culture. In some embodiments, the steviol, kaurene and/or steviol glycosides is recovered from the gas phase while in other embodiments, an organic layer or polymeric resin is added to the cell culture, and the kaurene, steviol and/or steviol glycosides is recovered from the organic layer or polymeric resin. In some embodiments, the steviol glycoside is selected from rebaudioside A, rebaudioside B, rebaudioside C, rebaudioside D, rebaudioside E, rebaudioside I or dulcoside A. In some embodiments, the terpenoid produced is steviobioside or stevioside. It should also be appreciated that in some embodiments, at least one enzymatic step, such as one or more glycosylation steps, are performed ex vivo.

Part of the invention is the production of the steviol glycoside that is then subject to further enzymatic conversion to Reb I. According to the current invention, the biosynthesis for the conversion of microbially produced steviol to a desired steviol glycosides (here Reb I) occurs when the diterpenoid steviol is converted from rubusoside and stevioside using multi-step chemical assembly of sugar moiety into the steviol backbone. In some embodiments, the biosynthesis for the conversion of Reb A to Reb I occurs by reacting Reb A with a glucose donor moiety in the presence of a recombinant polypeptide having glucosyltranserase activity. In some embodiments, the glucose donor moiety is generated in situ. In some embodiments, the glucose donor moiety is added to the reaction. For example, in some embodiments, an enzyme identified as UGT76G1 (SEQ ID NO: 10) can convert Reb A to Reb I. In some embodiments, a UGT76G1 mutant comprising a mutation L200A relative to SEQ ID NO: 9 (referred to herein as the “LA mutant,” the mutant having an amino acid sequence of SEQ ID NO: 1) can convert Reb A to Reb I. It was demonstrated herein that the LA mutant has increased activity in converting Reb A to Reb I.

### Biosynthesis of Steviol Glycosides

As described herein, the recombinant polypeptides of the present technology have UDP-glycosyltransferase activities and are useful for developing biosynthetic methods for preparing steviol glycosides that are either not present in nature or typically of low abundance in natural sources, such as rebaudioside I and rebaudioside M, respectively. The recombinant polypeptides of the present technology have UDP-glycosyltransferase activities, are useful for developing biosynthetic methods for preparing novel steviol glycosides, such as rebaudioside I and reaching the synthetic production of rebaudioside M.

The substrate can be any natural or synthetic compound capable of being converted into a steviol glycoside compound in a reaction catalyzed by one or more UDP glycosyltransferases. For example, the substrate can be natural stevia extract, steviol, steviol-13-O-glucoside, steviol-19-O-glucoside, steviol-1, 2-bioside, rubusoside, stevioside, rebaudioside A, rebaudioside G or rebaudioside E. The substrate can be a pure compound or a mixture of different compounds. Preferably, the substrate includes a compound selected from the group consisting of rubusoside, stevioside, steviol, rebaudioside A, rebaudioside E and combinations thereof.

The method described herein also provides a coupling reaction system in which the recombinant peptides described herein can function in combination with one or more additional enzymes to improve the efficiency or modify the outcome of the overall biosynthesis of steviol glycoside compounds. For example, the additional enzyme may regenerate the UDP-glucose needed for the glycosylation reaction by converting the UDP produced from the glycosylation reaction back to UDP-glucose (using, for example, sucrose as a donor of the glucose residue), thus improving the efficiency of the glycosylation reaction.

In another embodiment, the method of the subject technology further includes incubating a recombinant UDP-glycosyltransferase with the recombinant sucrose synthase, the substrate, and the recombinant polypeptide described herein. The recombinant UDP-glycosyltransferase can catalyze a different glycosylation reaction than the one catalyzed by the recombinant polypeptide of the subject technology.

Suitable UDP-glycosyltransferase includes any UGT known in the art as capable of catalyzing one or more reactions in the biosynthesis of steviol glycoside compounds, such as UGT85C2, UGT74G1, UGT76G1, or the functional homologs thereof.

Typically, in the in vitro method of the subject technology, UDP or UDP-Glucose is included in the buffer at a concentration of from about 0.2 mM to about 5 mM, preferably from about 0.5 mM to about 2 mM, more preferably from about 0.7 mM to about 1.5 mM. In an embodiment, when a recombinant sucrose synthase is included in the reaction, sucrose is also included in the buffer at a concentration of from about 100 mM to about 500 mM, preferably from about 200 mM to about 400 mM, more preferably from about 250 mM to about 350 mM. In some embodiments, in the in vitro method of the subject technology, the weight ratio of the recombinant polypeptide to the substrate, on a dry weight basis, is from about 1:100 to about 1:5, preferably from about 1:50 to about 1:10, more preferably from about 1:25 to about 1:15.

In some embodiments, the reaction temperature of the in vitro method is from about 20° C. to about 40° C., suitably from 25° C. to about 37° C., more suitably from 28° C. to about 32° C.

One with skill in the art will recognize that the steviol glycoside composition produced by the method described herein can be further purified and mixed with other steviol glycosides, flavors, or sweeteners to obtain a desired flavor or sweetener composition. For example, a composition enriched with rebaudioside M or Reb I produced as described herein can be mixed with a natural stevia extract containing rebaudioside A as the predominant steviol glycoside, or with other synthetic or natural steviol glycoside products to make a desired sweetener composition. Alternatively, a substantially purified steviol glycoside (e.g., rebaudioside I) obtained from the steviol glycoside composition described herein can be combined with other sweeteners, such as sucrose, maltodextrin, aspartame, sucralose, neotame, acesulfame potassium, and saccharin. The amount of steviol glycoside relative to other sweeteners can be adjusted to obtain a desired taste, as known in the art. The steviol glycoside compositions described herein (including rebaudioside D, rebaudioside A, rebaudioside I, rebaudioside M or a combination thereof) can be included in food products (such as beverages, soft drinks, ice cream, dairy products, confectioneries, cereals, chewing gum, baked goods, etc.), dietary supplements, medical nutrition, as well as pharmaceutical products.

One with skill in the art will recognize that the steviol glycoside composition produced by the method described herein can be further purified and mixed with other steviol glycosides, flavors, or sweeteners to obtain a desired flavor or sweetener composition. For example, a composition enriched with rebaudioside I produced as described herein can be mixed with a natural stevia extract containing rebaudioside A as the predominant steviol glycoside, or with other synthetic or natural steviol glycoside products to make a desired sweetener composition. Alternatively, a substantially purified steviol glycoside (e.g., rebaudioside I) obtained from the steviol glycoside composition described herein can be combined with other sweeteners, such as sucrose, maltodextrin, aspartame, sucralose, neotame, acesulfame potassium, and saccharin. The amount of steviol glycoside relative to other sweeteners can be adjusted to obtain a desired taste, as known in the art. The steviol glycoside composition described herein (including rebaudioside D, rebaudioside A, rebaudioside I, rebaudioside M or a combination thereof) can be included in food products (such as beverages, soft drinks, ice cream, dairy products, confectioneries, cereals, chewing gum, baked goods, etc.), dietary supplements, medical nutrition, as well as pharmaceutical products.

### Analysis of Sequence Similarity Using Identity Scoring

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT, FASTA, and TFASTA available as part of the GCG® Wisconsin Package® (Accelrys Inc., Burlington, Mass.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity is preferably determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, JOURNAL OF MOLECULAR BIOLOGY 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, ADVANCES IN APPLIED MATHEMATICS, 2:482-489, 1981, Smith et al., NUCLEIC ACIDS RESEARCH 11:2205-2220, 1983). The percent identity is most preferably determined using the “Best Fit” program.

Useful methods for determining sequence identity are also disclosed in the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. MOL. BIOL. 215:403-410 (1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

As used herein, the term “substantial percent sequence identity” refers to a percent sequence identity of at least about 70% sequence identity, at least about 80% sequence identity, at least about 85% identity, at least about 90% sequence identity, or even greater sequence identity, such as about 98% or about 99% sequence identity. Thus, one embodiment of the invention is a polynucleotide molecule that has at least about 70% sequence identity, at least about 80% sequence identity, at least about 85% identity, at least about 90% sequence identity, or even greater sequence identity, such as about 98% or about 99% sequence identity with a polynucleotide sequence described herein.

### Identity and Similarity

Identity is the fraction of amino acids that are the same between a pair of sequences after an alignment of the sequences (which can be done using only sequence information or structural information or some other information, but usually it is based on sequence information alone), and similarity is the score assigned based on an alignment using some similarity matrix. The similarity index can be any one of the following BLOSUM62, PAM250, or GONNET, or any matrix used by one skilled in the art for the sequence alignment of proteins.

Identity is the degree of correspondence between two sub-sequences (no gaps between the sequences). An identity of 25% or higher implies similarity of function, while 18-25% implies similarity of structure or function. Keep in mind that two completely unrelated or random sequences (that are greater than 100 residues) can have higher than 20% identity. Similarity is the degree of resemblance between two sequences when they are compared. This is dependent on their identity.

As is evident from the foregoing description, certain aspects of the present disclosure are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present disclosure.

Moreover, unless defined otherwise, all technical and scientific terms used herein have the same meaning as commonly understood by one of ordinary skill in the art to which the disclosure belongs. Although any methods and materials similar to or equivalent to or those described herein can be used in the practice or testing of the present disclosure, the preferred methods and materials are described above.

Although the foregoing invention has been described in some detail by way of illustration and example for purposes of understanding, it will be apparent to those skilled in the art that certain changes and modifications may be practiced. Therefore, the description and examples should not be construed as limiting the scope of the invention, which is delineated by the appended claims.

### Mutant Enzymes

Based on the crystal structure of UGT76G1, a series of circular permutations and a set of mutations were designed and tested for their function. After activity screening, one version of UGT76G1 circular permutation mutant, CP1, was found to be significantly active. Broadly speaking, CP1 is a variant of UGT76G1 with its domains switched and identified mutation sites. CP1 demonstrated significant activity in terms of glucosylation of the steviol core. When a linker was inserted into the CP1 mutant to generate a second mutant CP2, CP2 was found to show similar activity as the CP1 mutant.

Based on modeling analysis of UGT76G1, mutation sites for the UGT76G1 enzyme were selected and tested for their activities in the bioconversion of Reb A to Reb I. After a series of such mutations, a handful of mutants were identified with the desired enzymatic function with regard to glycosylation activity and ornamentation of the steviol core. Then a genetically modified microbe was developed, which is capable of converting Reb A to Reb I. For example, one UGT76G1 mutant (L200A, referred to herein as the LA mutant) was found to have extremely high enzymatic activity for the bioconversion of Reb A to Reb I. The LA mutant includes one mutation site (L200A) from the UGT76G1 sequence. In some embodiments, the LA mutant comprises the amino acid sequence of SEQ ID NO: 1.

### EXAMPLES

### Example 1

### Enzymatic Activity Screening of UGT Enzymes

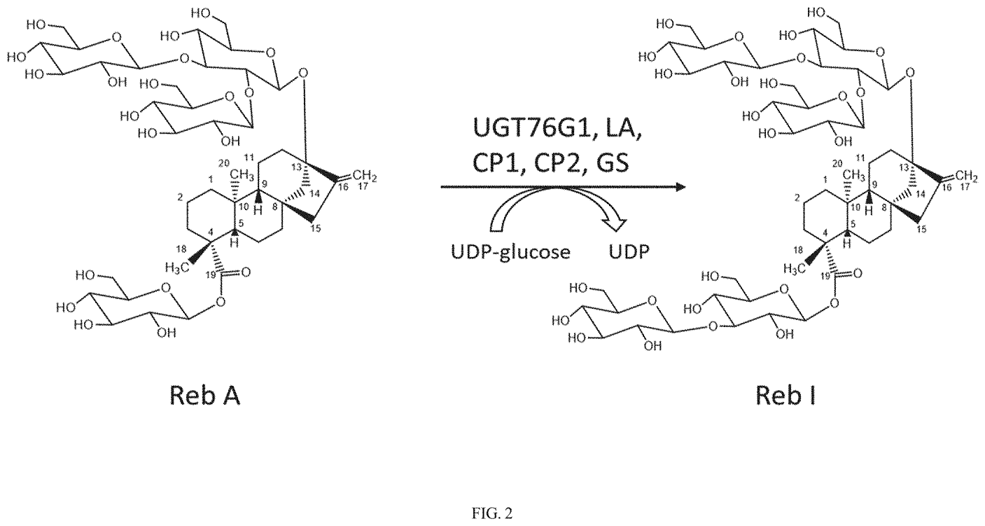

The majority of the steviol glycosides are formed by several glycosylation reactions of steviol, which typically are catalyzed by the UDP-glycosyltransferases (UGTs) using uridine 5′-diphosphoglucose (UDP-glucose) as a donor of the sugar. In plants, UGTs are a very divergent group of enzymes that transfer a glucose residue from UDP-glucose to steviol. For example, glycosylation of the C-3′ of the C-13-O-glucose of stevioside yields rebaudioside A (UGT76G1). In order to produce rebaudioside I (Reb I) from Reb A, the UGT needs to transfer a glucose residue from UDP-glucose to Reb A, glycosylating of the C-3′ position of the C-19-O-glucose of Reb A (FIG. 2). In order to identify the specific UGT enzyme for Reb I production from Reb A, UGT76G1 and related mutants were chosen based on protein structure for activity screening.

Full-length DNA fragments of all candidate UGT genes were commercially synthesized. Almost all codons of the cDNA were changed to those preferred for E. coli (Genscript, NJ). The synthesized DNA was cloned into a bacterial expression vector pETite N-His SUMO Kan Vector (Lucigen).

Each expression construct was transformed into E. coli BL21 (DE3), which was subsequently grown in LB media containing 50 μg/mL kanamycin at 37° C. until reaching an OD600 of 0.8-1.0. Protein expression was induced by addition of 1 mM isopropyl β-D-1-thiogalactopyranoside (IPTG) and the culture was further grown at 16 ° C. for 22 hr. Cells were harvested by centrifugation (3,000×g; 10 min; 4° C.). The cell pellets were collected and were either used immediately or stored at −80° C.

The cell pellets typically were re-suspended in lysis buffer (50 mM potassium phosphate buffer, pH 7.2, 25 ug/ml lysozyme, 5 ug/ml DNase I, 20 mM imidazole, 500 mM NaCl, 10% glycerol, and 0.4% Triton X-100). The cells were disrupted by sonication under 4° C., and the cell debris was clarified by centrifugation (18,000×g; 30 min). Supernatant was loaded to an equilibrated (equilibration buffer: 50 mM potassium phosphate buffer, pH 7.2, 20 mM imidazole, 500 mM NaCl, 10% glycerol) Ni-NTA (Qiagen) affinity column. After loading of protein sample, the column was washed with equilibration buffer to remove unbound contaminant proteins. The His-tagged beta-glucosidase recombinant polypeptides were eluted by equilibration buffer containing 250 mM imidazole.

The purified candidate UGT recombinant polypeptides were assayed for glycosyltransferase activity by using various steviol glycosides as substrate. Typically, the recombinant polypeptide (20 μg) was tested in a 200 μl in vitro reaction system. The reaction system contains 50 mM potassium phosphate buffer, pH 7.2, 3mM MgCl₂, 1 mg/ml steviol glycoside and 3mM UDP-glucose. The reaction was performed at 30-37° C. and 50 ul reaction was terminated by adding 200 μL 1-butanol at various time points. The samples were extracted three times with 200 μL 1-butanol. The pooled fraction was dried and dissolved in 100 μL 80% methanol for high-performance liquid chromatography (HPLC) analysis.

HPLC analysis was then performed using a Dionex UPLC ultimate 3000 system (Sunnyvale, Calif.), including a quaternary pump, a temperature-controlled column compartment, an auto sampler and a UV absorbance detector. A Synergi Hydro-RP column (Phenomenex) with guard column was used for the characterization of steviol glycosides in the pooled samples. The detection wavelength used in the HPLC analysis was 210nm.

After activity screening, several enzymes having UDP-glycosyltransferase activity were identified for Reb I production.

UGT76G1 enzyme can convert Reb A to Reb I. After screening various mutants and variants, a particular variant, LA, which has only one mutation (L200A) compared to the wild type UGT76G1, unexpectedly showed significantly higher activity in converting Reb A to Reb I as described in more details in Example 2.

### Example 2

### Enzymatic Bioconversion of Reb A to Reb I

Circular permutation analysis is a powerful tool to develop useful or valuable enzymes (PLoS computational Biology, 2012, 8(3) e1002445; BIOINFORMATICS, 2015, (3)). Based on the crystal structure of UGT76G1, a series of circular permutations were designed. After performing activity screening, one version of circular permutation (“CP1”) was found to have higher activity for Reb I production compared to WT UGT76G1. A linker (YKDDSGYSSSYAAAAGM (SEQ ID NO: 15)) was inserted between the C-terminal and the N terminal of CP1 to generate a second mutant (“CP2”), which also has higher activity than WT UGT76G1 for Reb I production.

Based on modeling analysis of UGT76G1, mutation sites were selected to enhance enzymatic activity. After enzymatic activity screening of the various mutants generated, it was unexpectedly found that one mutant, LA (an L200A mutant of UGT76G1), showed a significant increase in its enzymatic activity in converting Reb A to Reb I, especially when compared to the wild type UGT76G1.

To confirm the conversion of rebaudioside A to rebaudioside I in vitro, the selected UGT enzymes were assayed using Reb A as the steviol glycoside substrate. The reaction system contained 50 mM potassium phosphate buffer (pH 7.2), 3 mM MgCl₂, 1 mg/ml stevioside, 3mM UDP-glucose, and enzyme (20 ug/200 ul reaction). The reaction was performed at 37° C. and terminated by adding 1-butanol. The samples were extracted three times with 1-butanol. The pooled fraction was dried and dissolved in 80% methanol for high-performance liquid chromatography (HPLC) analysis. HPLC analysis was performed as above description.

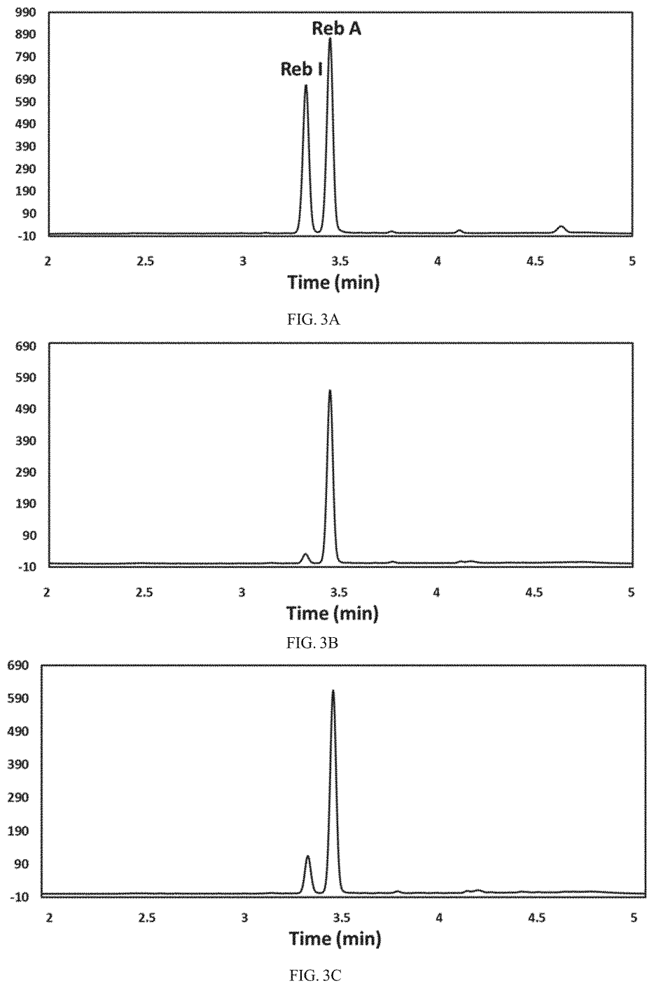

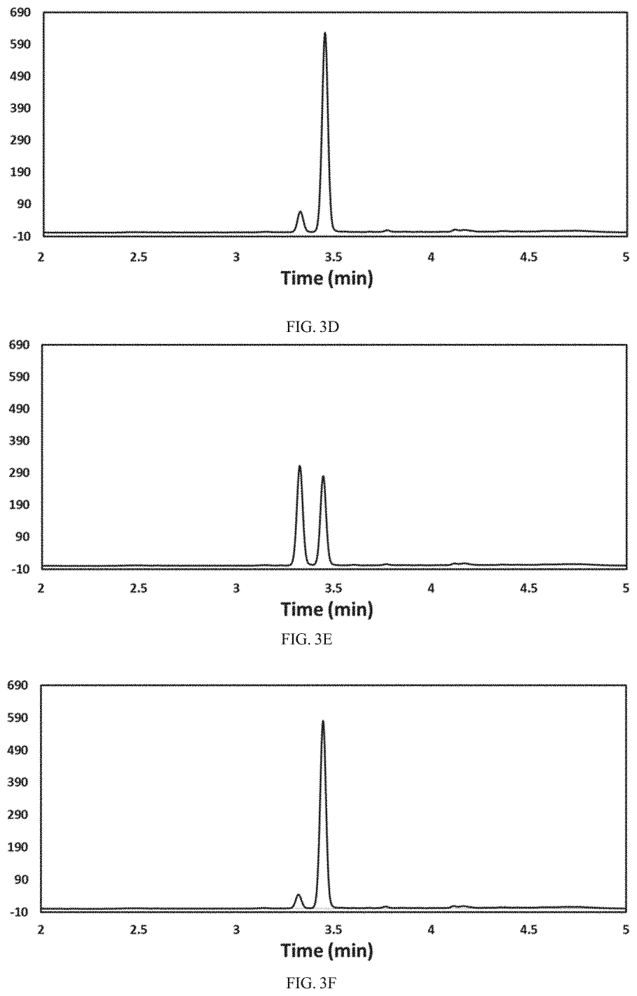

As shown in FIGS. 3A-3F, UGT76G1 can convert Reb A to Reb I (FIG. 3B). Both CP1 (FIG. 3C) and CP2 (FIG. 3D) mutants have higher enzymatic activity than UGT76G1. CP2 has lower activity than CP1 indicating that the selected linker between the N-terminal and C-terminal affects the enzymatic activity. In order to identify the mutants having high enzymatic activity, several site mutants were generated based on UGT76G1 crystal structure. After activity screening, mutants that have higher activity than UGT76G1 were found. The LA mutant (L200A) has the highest activity among all the tested mutants. As shown in FIG. 3E, the peaks corresponding to Reb I and Reb A, respectively, show that more than 50% of the Reb A was consumed and converted to Reb I when LA was used. By comparison, when any of UGT76G1, CP1 and CP2 was used, less than 15% of Reb A was converted to Reb I (FIGS. 3B-3D). The UGT76G1-AtSUS1 fusion enzyme (GS) also showed higher activity for bioconversion of Reb A to Reb I (FIG. 3F) compared to UGT76G1, although not as significant as LA, CP1 or CP2.

In a coupling system in which both a UGT enzyme and a sucrose synthase (SUS) are present, UDP-glucose can be regenerated from UDP and sucrose, which allows for omitting the addition of extra UDP-glucose to the reaction mixture. Suitable sucrose synthases (SUS) can be for example, an Arabidopsis sucrose synthase 1; an Arabidopsis sucrose synthase 3; and a Vigna radiate sucrose synthase.

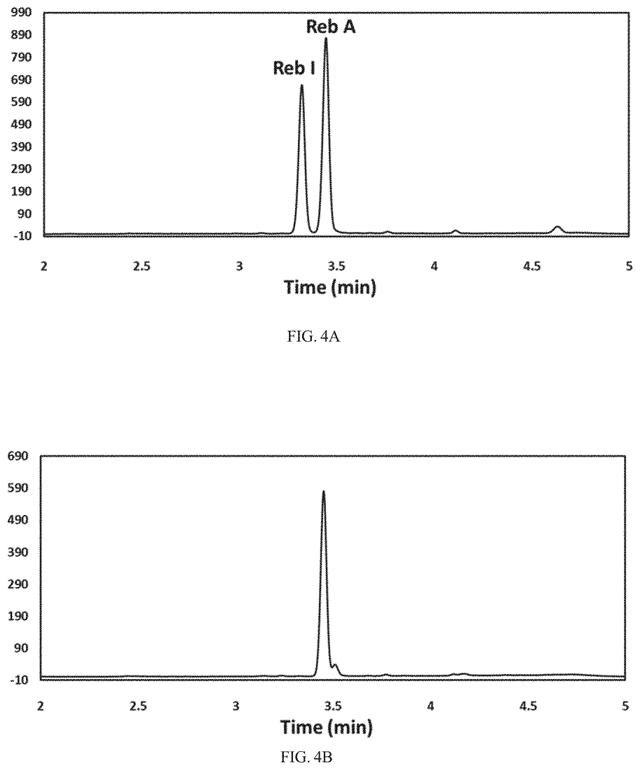

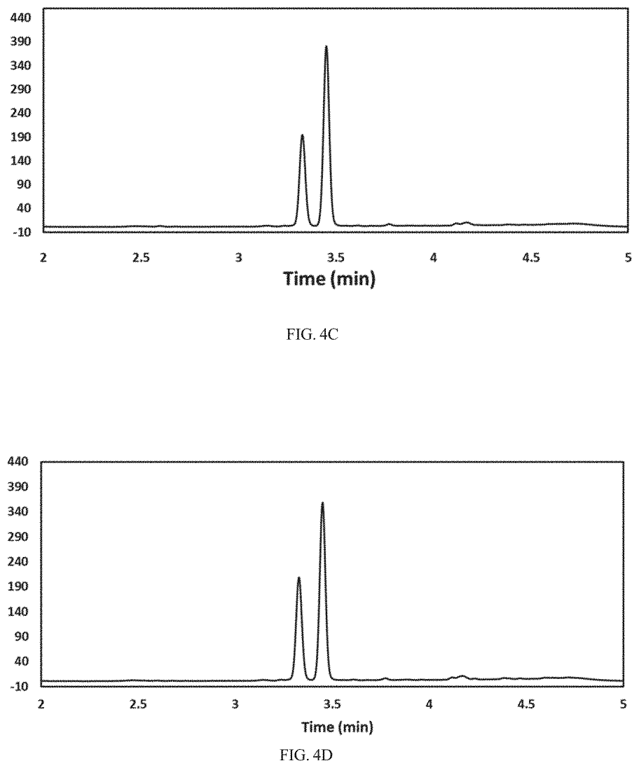

In another aspect, UDP-glycosyltransferase fusion enzyme can be used in the methods. A particularly suitable UDP-glycosyltransferase fusion enzyme can be a UGT-SUS fusion enzyme. The UDP-glycosyltransferase can be a UDP-glycosyltransferase fusion enzyme that includes a UDP-glycosyltransferase domain coupled to a sucrose synthase domain. In particular, the UDP-glycosyltransferase fusion enzyme includes a UDP-glycosyltransferase domain coupled to a sucrose synthase domain. Additionally, the UGT-SUS fusion enzyme has sucrose synthase activity, and thus, can regenerate UDP-glucose from UDP and sucrose. A particularly suitable UGT-SUS fusion enzyme can be, for example, a UGT76G1-AtSUS1 fusion enzyme (named as: “GS”). GS fusion enzyme can convert Reb A to Reb I in addition of UDP and sucrose (FIGS. 4A-4D). As shown in FIGS. 4A-4D, UGT76G1 cannot convert Reb A to Reb I in UDP addition (FIG. 4B). However, both of GS fusion enzyme (FIG. 4D) and combination of UGT76G1 and AtSUS1 enzyme system (FIG. 4C) can produce Reb I from Reb A indicating the UDPG regeneration by AtSUS1 sucrose synthase. It was also found that GS fusion enzyme has higher enzymatic activity than the combination of UGT76G1 and AtSUS1 enzyme in the same reaction system. Sucrose synthases (SUS) catalyze the conversion of the UDP to UDP-glucose in the presence of sucrose. Thus, for a glycosylation reaction utilizing UDP-glucose (such as those catalyzed by the UGTs), SUS can be used to re-generate UDP-glucose from UDP, enhancing the efficiency of such reaction.

### Example 3

### NMR Analysis the Structure of Produced Reb I

The produced Reb I compound was purified by semi preparative chromatography as described above.

The molecular formula of Reb I has been deduced as C₅₀H₈₀O₂₈ on the basis of its positive High Resolution Mass Spectrum (HRMS) which showed an adduct ions corresponding to [M+NH_{4]}⁺ and [M+Na]⁺ at m/z 1146.5281 and 1151.4839 respectively , and this composition was supported by the ¹³C NMR spectral data. The ¹H NMR spectrum of Reb I showed the presence of two methyl singlets at δ 1.22, and 1.26; nine methylene and two methine protons between δ 0.75-2.59; and two singlets corresponding to an exocyclic double bond at δ 5.01 and 5.65; similar to the ent-kaurane diterpenoids isolated earlier from S. rebaudiana. The basic skeleton of ent-kaurane diterpenoids was supported by the key TOCSY (H-1/H-2; H-2/H-3; H-5/H-6; H-6/H-7; H-9/H-11; H-11/H-12) and HMBC (H-1/C-2, C-10; H-3/C-1, C-2, C-4, C-5,

C-18, C-19; H-5/C-4, C-6, C-7, C-9, C-10, C-18, C-19, C-20; H-9/C-8, C-10, C-11, C-12, C-14, C-15; H-14/C-8, C-9, C-13, C-15, C-16 and H-17/C-13, C-15, C-16) correlations.

Further, the ¹H NMR spectrum of Reb I showed anomeric protons as doublets at δ 5.03, 5.24, 5.34, 5.53, and 6.12 suggesting the presence of five sugar units in its structure. Acid hydrolysis of Reb I with 5% H₂SO₄ afforded D-glucose which was identified by direct comparison with authentic sample by TLC suggested the presence of six glucopyranosyl moieties in its molecular structure. The configuration of D-glucose was identified by preparing its corresponding thiocarbamoyl-thiazolidine carboxylate derivative with L-cysteine methyl ester and O-tolyl isothiocyanate, and in comparison, of its retention time with the standard sugars as described in the literature comparison. Enzymatic hydrolysis of Reb I furnished an aglycone which was identified as steviol by comparison of ¹H NMR and co-TLC with standard compound. The ¹H and ¹³C NMR values for compound Reb I were assigned on the basis of COSY, TOCSY, HMQC, HMBC and ROESY data.

TABLE 2
¹H and ¹³C NMR spectral data (chemical shifts and coupling
constants) for produced Rebaudioside I^{a-c}.

| Position | ¹H NMR | ¹³C NMR |
|---|---|---|
| 1 | 0.75 (dt, J = 3.1, 12.8, 1H), 1.77 (m, 1H) | 41.0 |
| 2 | 1.45 (m, 1H), 2.20 (m, 1H) | 19.7 |
| 3 | 1.00 (m, 1H), 2.32 d (12.8) | 38.8 |
| 4 | | 44.6 |
| 5 | 1.02 (d, J = 12.8, 1H) | 57.5 |
| 6 | 1.89 (m, 1H), 2.28 (m, 1H) | 22.5 |
| 7 | 1.33 (m, 1H) | 42.0 |
| 8 | | 42.6 |
| 9 | 0.88 (d, J = 7.6, 1H) | 54.4 |
| 10 | | 40.1 |
| 11 | 1.64 (m, 1H), 1.72 (m, 1H) | 20.8 |
| 12 | 1.95 (m, 1H), 2.33 (m, 1H) | 37.6 |
| 13 | | 86.9 |
| 14 | 1.76 (m, 1H), 2.59 (d, J = 11.2, 1H) | 44.3 |
| 15 | 2.06 ( br q, J = 8.5, 1H) | 48.0 |
| 16 | | 154.3 |
| 17 | 5.01 (s, 1H), 5.65 (s, 1H) | 105.0 |
| 18 | 1.22 (s, 3H) | 28.7 |
| 19 | | 177.2 |
| 20 | 1.26 (s, 3H) | 16.0 |
| 1′ | 6.12 (d, J = 8.1, 1H) | 95.6 |
| 2′ | 4.16 (m, 1H) | 72.5 |
| 3′ | 4.24 (m, 1H) | 89.8 |
| 4′ | 4.21 (m, 1H) | 69.6 |
| 5′ | 3.90 (m, 1H) | 78.3 |
| 6′ | 4.25 (m, 1H), 4.39 (m, 1H) | 62.6 |
| 1″ | 5.03 (d, J = 7.8, 1H) | 98.3 |
| 2″ | 4.30 (m, 1H) | 80.9 |
| 3″ | 4.16 (m, 1H) | 88.1 |
| 4″ | 4.08 (m, 1H) | 70.4 |
| 5″ | 3.77 (m, 1H) | 77.9 |
| 6″ | 4.18 (m, 1H), 4.48 (m, 1H) | 62.7 |
| 1′′′ | 5.53 (d, J = 7 .8, 1H) | 104.9 |
| 2′′′ | 4.18 (m, 1H) | 76.5 |
| 3′′′ | 4.29 (m, 1H) | 78.5 |
| 4′′′ | 4.23 (m, 1H) | 72.8 |
| 5′′′ | 3.94 (m, 1H) | 77.8 |
| 6′′′ | 4.36 (m, 1H), 4.54 (m, 1H) | 62.9 |
| 1′′′′ | 5.34 (d, J = 7.8, 1H) | 105.0 |
| 2′′′′ | 3.98 (m, 1H) | 75.6 |
| 3′′′′ | 4.14 (m, 1H) | 78.8 |
| 4′′′′ | 4.08 (m, 1H) | 71.8 |
| 5′′′′ | 4.12 (m, 1H) | 78.5 |
| 6′′′′ | 4.20 (m, 1H), 4.52 (m, 1H) | 62.0 |
| 1′′′′′ | 5.24 (d, J = 7.8, 1H) | 105.4 |
| 2′′′′′ | 4.06 (m, 1H) | 75.6 |
| 3′′′′′ | 4.31 (m, 1H) | 78.8 |
| 4′′′′′ | 4.11 (m, 1H) | 71.9 |
| 5′′′′′ | 4.07 (m, 1H) | 78.9 |
| 6′′′′′ | 4.32 (m, 1H), 4.52 (m, 1H) | 63.4 |

^{a}assignments made on the basis of COSY, TOCSY, HMQC, HMBC and ROESY correlations;
^{b}Chemical shift values are in δ (ppm);
^{c}Coupling constants are in Hz.

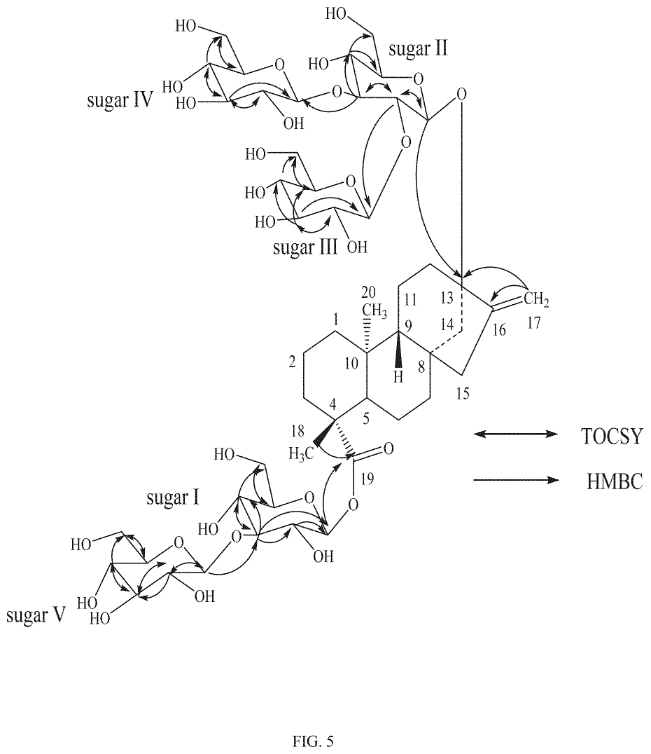

A close study of the ¹H and ¹³C NMR values of Reb I together with enzymatic and acid hydrolysis experiments suggested that compound Reb I is also a steviol glycoside with three glucosyl moieties residues that are attached at the C-13 hydroxyl as a 2,3-branched glucotriosyl substituent and another glucosyl moiety in the form of an ester at C-19 position leaving the identification of additional glucosyl unit. The downfield shift for both the ¹H and ¹³C chemical shifts at 3′-position of sugar I suggested the additional β-D-glucosyl moiety at this position which was supported by the key HMBC correlations: H-1′/C-19, C-2′; H-2′/C-1′, C-3′ and H-3′/C-1′, C-2′ and C-4′. The large coupling constants observed for the five anomeric protons at δ 5.03 (d, J=7.8 Hz), 5.24 (d, J=7.8 Hz), 5.34 (d, J=7.8 Hz), 5.53 (d, J=7.8 Hz) and 6.12 (d, J=8.1 Hz), suggested their β-orientation as reported for steviol glycosides. The structure of Reb I was further supported by the key TOCSY, and HMBC correlations as shown in FIG. 5.

Based on the results from chemical and spectral studies, Reb I was assigned as 13-[(2-O-β-D-glucopyranosyl-3-O-β-D-glucopyranosyl-β-D-glucopyranosyl)oxy] ent-kaur-16-en-19-oic acid-(3-O-β-D-glucopyranosyl-β-D-glucopyranosyl) ester and its spectral data are in consistent with the structural data of Reabudioside I reported in the literature.

A solution of produced Reb I (3 mg) in MeOH (10 ml) was added 3 ml of 5% H₂SO₄ and the mixture was refluxed for 16 hours. The reaction mixture was then neutralized with saturated sodium carbonate and extracted with ethyl acetate (EtOAc) (2×25 ml) to give an aqueous fraction containing sugars and an EtOAc fraction containing the aglycone part. The aqueous phase was concentrated and compared with standard sugars using the TLC systems EtOAc/n-butanol/water (2:7:1) and CH₂Cl₂/MeOH/water (10:6:1) [6-8]; the sugars were identified as D-glucose.

Produced Reb I (500 μg) was hydrolyzed with 0.5 M HCl (0.5 mL) for 1.5 h. After cooling, the mixture was passed through an Amberlite IRA400 column and the eluate was lyophilized. The residue was dissolved in pyridine (0.25 mL) and heated with L-cysteine methyl ester HCl (2.5 mg) at 60° C. for 1.5 h, and then O-tolyl isothiocyanate (12.5 uL) was added to the mixture and heated at 60° C. for an additional 1.5 h. HPLC analysis of the reaction mixture was performed by a Phenomenex Luna column [C18, 150×4.6 mm (5 u)] using the mobile phase 25% acetonitrile-0.2% TFA water, 1 mL/min under UV detection at 250 nm. The sugar was identified as D-glucose (tR, 12.64) [authentic samples, D-glucose (tR, 12.54) and L-glucose (tR, 11.42 min)] [9].

Produced Reb I (1 mg) was dissolved in 2.5 ml of 0.1 M sodium acetate buffer by maintaining pH at 4.5 and 50 μL of crude pectinase from Aspergillus niger (Sigma-Aldrich) was added. The mixture was stirred at 50° C. for 48 hr and the product precipitated out during the reaction was filtered and then crystallized. The resulting product obtained was identified as steviol by comparison of their ¹H NMR spectral data and co-TLC.

The complete ¹H and ¹³C NMR spectral data for 13-[(2-O-β-D-glucopyranosyl-3-O-β-D-glucopyranosyl-β-D-glucopyranosyl)oxy] ent-kaur-16-en-19-oic acid-(3-O-β-D-glucopyranosyl-β-D-glucopyranosyl) ester (Rebaudioside I) produced by enzymatic bioconversion has been assigned on the basis of extensive 1D and 2D NMR as well as high resolution mass spectral data. The structure of Rebaudioside I was further supported by acid and enzymatic hydrolysis studies.

Sequences of Interest
UGT76G1 L200A mutant (LA): Amino Acid Sequence:
(SEQ ID NO: 1)
MENKTETTVRRRRRIILFPVPFQGHINPILQLANV
LYSKGFSITIFHTNFNKPKTSNYPHFTFRFILDND
PQDERISNLPTHGPLAGMRIPIINEHGADELRREL
ELLMLASEEDEEVSCLITDALWYFAQSVADSLNLR
RLVLMTSSLFNFHAHVSLPQFDELGYLDPDDKTRL
EEQASGFPMLKVKDIKSAYSNWQIAKEILGKMIKQ
TKASSGVIWNSFKELEESELETVIREIPAPSFLIP
LPKHLTASSSSLLDHDRTVFQWLDQQPPSSVLYVS
FGSTSEVDEKDFLEIARGLVDSKQSFLWVVRPGFV
KGSTWVEPLPDGFLGERGRIVKWVPQQEVLAHGAI
GAFWTHSGWNSTLESVCEGVPMIFSDFGLDQPLNA
RYMSDVLKVGVYLENGWERGEIANAIRRVMVDEEG
EYIRQNARVLKQKADVSLMKGGSSYESLESLVSYI
SSL
UGT76G1 L200A mutant (LA): DNA Sequence:
(SEQ ID NO: 2)
ATGGAGAATAAGACAGAAACAACCGTAAGACGGAG
GCGGAGGATTATCTTGTTCCCTGTACCATTTCAGG
GCCATATTAATCCGATCCTCCAATTAGCAAACGTC
CTCTACTCCAAGGGATTTTCAATAACAATCTTCCA
TACTAACTTTAACAAGCCTAAAACGAGTAATTATC
CTCACTTTACATTCAGGTTCATTCTAGACAACGAC
CCTCAGGATGAGCGTATCTCAAATTTACCTACGCA
TGGCCCCTTGGCAGGTATGCGAATACCAATAATCA
ATGAGCATGGAGCCGATGAACTCCGTCGCGAGTTA
GAGCTTCTCATGCTCGCAAGTGAGGAAGACGAGGA
AGTTTCGTGCCTAATAACTGATGCGCTTTGGTACT
TCGCCCAATCAGTCGCAGACTCACTGAATCTACGC
CGTTTGGTCCTTATGACAAGTTCATTATTCAACTT
TCACGCACATGTATCACTGCCGCAATTTGACGAGT
TGGGTTACCTGGACCCGGATGACAAAACGCGATTG
GAGGAACAAGCGTCGGGCTTCCCCATGCTGAAAGT
CAAAGATATTAAGAGCGCTTATAGTAATTGGCAAA
TTGCGAAAGAAATTCTCGGAAAAATGATAAAGCAA
ACCAAAGCGTCCTCTGGAGTAATCTGGAACTCCTT
CAAGGAGTTAGAGGAATCTGAACTTGAAACGGTCA
TCAGAGAAATCCCCGCTCCCTCGTTCTTAATTCCA
CTACCCAAGCACCTTACTGCAAGTAGCAGTTCCCT
CCTAGATCATGACCGAACCGTGTTTCAGTGGCTGG
ATCAGCAACCCCCGTCGTCAGTTCTATATGTAAGC
TTTGGGAGTACTTCGGAAGTGGATGAAAAGGACTT
CTTAGAGATTGCGCGAGGGCTCGTGGATAGCAAAC
AGAGCTTCCTGTGGGTAGTGAGACCGGGATTCGTT
AAGGGCTCGACGTGGGTCGAGCCGTTGCCAGATGG
TTTTCTAGGGGAGAGAGGGAGAATCGTGAAATGGG
TTCCACAGCAAGAGGTTTTGGCTCACGGAGCTATA
GGGGCCTTTTGGACCCACTCTGGTTGGAATTCTAC
TCTTGAAAGTGTCTGTGAAGGCGTTCCAATGATAT
TTTCTGATTTTGGGCTTGACCAGCCTCTAAACGCT
CGCTATATGTCTGATGTGTTGAAGGTTGGCGTGTA
CCTGGAGAATGGTTGGGAAAGGGGGGAAATTGCCA
ACGCCATACGCCGGGTAATGGTGGACGAGGAAGGT
GAGTACATACGTCAGAACGCTCGGGTTTTAAAACA
AAAAGCGGACGTCAGCCTTATGAAGGGAGGTAGCT
CCTATGAATCCCTAGAATCCTTGGTAAGCTATATA
TCTTCGTTATAA
UGT76G1 CP1 mutant (CP1): Amino Acid Sequence:
(SEQ ID NO: 3)
MNWQILKEILGKMIKQTKASSGVIWNSFKELEESE
LETVIREIPAPSFLIPLPKHLTASSSSLLDHDRTV
FQWLDQQPPSSVLYVSFGSTSEVDEKDFLEIARGL
VDSKQSFLWVVRPGFVKGSTWVEPLPDGFLGERGR
IVKWVPQQEVLAHGAIGAFWTHSGWNSTLESVCEG
VPMIFSDFGLDQPLNARYMSDVLKVGVYLENGWER
GEIANAIRRVMVDEEGEYIRQNARVLKQKADVSLM
KGGSSYESLESLVSYISSLENKTETTVRRRRRIIL
FPVPFQGHINPILQLANVLYSKGFSITIFHTNFNK
PKTSNYPHFTFRFILDNDPQDERISNLPTHGPLAG
MRIPIINEHGADELRRELELLMLASEEDEEVSCLI
TDALWYFAQSVADSLNLRRLVLMTSSLFNFHAHVS
LPQFDELGYLDPDDKTRLEEQASGFPMLKVKDIKS
AYS
UGT76G1 CP1 mutant (CP1): DNA Sequence:
(SEQ ID NO: 4)
ATGAACTGGCAAATCCTGAAAGAAATCCTGGGTAA
AATGATCAAACAAACCAAAGCGTCGTCGGGCGTTA
TCTGGAACTCCTTCAAAGAACTGGAAGAATCAGAA
CTGGAAACCGTTATTCGCGAAATCCCGGCTCCGTC
GTTCCTGATTCCGCTGCCGAAACATCTGACCGCGA
GCAGCAGCAGCCTGCTGGATCACGACCGTACGGTC
TTTCAGTGGCTGGATCAGCAACCGCCGTCATCGGT
GCTGTATGTTTCATTCGGTAGCACCTCTGAAGTCG
ATGAAAAAGACTTTCTGGAAATCGCTCGCGGCCTG
GTGGATAGTAAACAGTCCTTCCTGTGGGTGGTTCG
TCCGGGTTTTGTGAAAGGCAGCACGTGGGTTGAAC
CGCTGCCGGATGGCTTCCTGGGTGAACGCGGCCGT
ATTGTCAAATGGGTGCCGCAGCAAGAAGTGCTGGC
ACATGGTGCTATCGGCGCGTTTTGGACCCACTCTG
GTTGGAACAGTACGCTGGAATCCGTTTGCGAAGGT
GTCCCGATGATTTTCAGCGA
TTTTGGCCTGGACCAGCCGCTGAATGCCCGCTATA
TGTCTGATGTTCTGAAAGTCGGTGTGTACCTGGAA
AACGGTTGGGAACGTGGCGAAATTGCGAATGCCAT
CCGTCGCGTTATGGTCGATGAAGAAGGCGAATACA
TTCGCCAGAACGCTCGTGTCCTGAAACAAAAAGCG
GACGTGAGCCTGATGAAAGGCGGTAGCTCTTATGA
ATCACTGGAATCGCTGGTTAGCTACATCAGTTCCC
TGGAAAATAAAACCGAAACCACGGTGCGTCGCCGT
CGCCGTATTATCCTGTTCCCGGTTCCGTTTCAGGG
TCATATTAACCCGATCCTGCAACTGGCGAATGTTC
TGTATTCAAAAGGCTTTTCGATCACCATCTTCCAT
ACGAACTTCAACAAACCGAAAACCAGTAACTACCC
GCACTTTACGTTCCGCTTTATTCTGGATAACGACC
CGCAGGATGAACGTATCTCCAATCTGCCGACCCAC
GGCCCGCTGGCCGGTATGCGCATTCCGATTATCAA
TGAACACGGTGCAGATGAACTGCGCCGTGAACTGG
AACTGCTGATGCTGGCCAGTGAAGAAGATGAAGAA
GTGTCCTGTCTGATCACCGACGCACTGTGGTATTT
CGCCCAGAGCGTTGCAGATTCTCTGAACCTGCGCC
GTCTGGTCCTGATGACGTCATCGCTGTTCAATTTT
CATGCGCACGTTTCTCTGCCGCAATTTGATGAACT
GGGCTACCTGGACCCGGATGACAAAACCCGTCTGG
AAGAACAAGCCAGTGGTTTTCCGATGCTGAAAGTC
AAAGACATTAAATCCGCCTATTCGTAA
UGT76G1 CP2 mutant (CP2): Amino Acid Sequence:
(SEQ ID NO: 5)
MNWQILKEILGKMIKQTKASSGVIWNSFKELEESE
LETVIREIPAPSFLIPLPKHLTASSSSLLDHDRTV
FQWLDQQPPSSVLYVSFGSTSEVDEKDFLEIARGL
VDSKQSFLWVVRPGFVKGSTWVEPLPDGFLGERGR
IVKWVPQQEVLAHGAIGAFWTHSGWNSTLESVCEG
VPMIFSDFGLDQPLNARYMSDVLKVGVYLENGWER
GEIANAIRRVMVDEEGEYIRQNARVLKQKADVSLM
KGGSSYESLESLVSYISSLYKDDSGYSSSYAAAAG
MENKTETTVRRRRRIILFPVPFQGHINPILQLANV
LYSKGFSITIFHTNFNKPKTSNYPHFTFRFILDND
PQDERISNLPTHGPLAGMRIPIINEHGADELRREL
ELLMLASEEDEEVSCLITDALWYFAQSVADSLNLR
RLVLMTSSLFNFHAHVSLPQFDELGYLDPDDKTRL
EEQASGFPMLKVKDIKSAYS
UGT76G1 CP2 mutant (CP2): DNA Sequence:
(SEQ ID NO: 6)
ATGAACTGGCAAATCCTGAAAGAAATCCTGGGTAA
AATGATCAAACAAACCAAAGCGTCGTCGGGCGTTA
TCTGGAACTCCTTCAAAGAACTGGAAGAATCAGAA
CTGGAAACCGTTATTCGCGAAATCCCGGCTCCGTC
GTTCCTGATTCCGCTGCCGAAACATCTGACCGCGA
GCAGCAGCAGCCTGCTGGATCACGACCGTACGGTC
TTTCAGTGGCTGGATCAGCAACCGCCGTCATCGGT
GCTGTATGTTTCATTCGGTAGCACCTCTGAAGTCG
ATGAAAAAGACTTTCTGGAAATCGCTCGCGGCCTG
GTGGATAGTAAACAGTCCTTCCTGTGGGTGGTTCG
TCCGGGTTTTGTGAAAGGCAGCACGTGGGTTGAAC
CGCTGCCGGATGGCTTCCTGGGTGAACGCGGCCGT
ATTGTCAAATGGGTGCCGCAGCAAGAAGTGCTGGC
ACATGGTGCTATCGGCGCGTTTTGGACCCACTCTG
GTTGGAACAGTACGCTGGAATCCGTTTGCGAAGGT
GTCCCGATGATTTTCAGCGATTTTGGCCTGGACCA
GCCGCTGAATGCCCGCTATATGTCTGATGTTCTGA
AAGTCGGTGTGTACCTGGAAAACGGTTGGGAACGT
GGCGAAATTGCGAATGCCATCCGTCGCGTTATGGT
CGATGAAGAAGGCGAATACATTCGCCAGAACGCTC
GTGTCCTGAAACAAAAAGCGGACGTGAGCCTGATG
AAAGGCGGTAGCTCTTATGAATCACTGGAATCGCT
GGTTAGCTACATCAGTTCCCTGTACAAAGATGACA
GCGGTTATAGCAGCAGCTATGCGGCGGCGGCGGGT
ATGGAAAATAAAACCGAAACCACGGTGCGTCGCCG
TCGCCGTATTATCCTGTTCCCGGTTCCGTTTCAGG
GTCATATTAACCCGATCCTGCAACTGGCGAATGTT
CTGTATTCAAAAGGCTTTTCGATCACCATCTTCCA
TACGAACTTCAACAAACCGAAAACCAGTAACTACC
CGCACTTTACGTTCCGCTTTATTCTGGATAACGAC
CCGCAGGATGAACGTATCTCCAATCTGCCGACCCA
CGGCCCGCTGGCCGGTATGCGCATTCCGATTATCA
ATGAACACGGTGCAGATGAACTGCGCCGTGAACTG
GAACTGCTGATGCTGGCCAGTGAAGAAGATGAAGA
AGTGTCCTGTCTGATCACCGACGCACTGTGGTATT
TCGCCCAGAGCGTTGCAGATTCTCTGAACCTGCGC
CGTCTGGTCCTGATGACGTCATCGCTGTTCAATTT
TCATGCGCACGTTTCTCTGCCGCAATTTGATGAAC
TGGGCTACCTGGACCCGGATGACAAAACCCGTCTG
GAAGAACAAGCCAGTGGTTTTCCGATGCTGAAAGT
CAAAGACATTAAATCCGCCTATTCGTAA
UGT76G1-AtSUS1 fusion enzyme (GS):
Amino Acid Sequence:
(SEQ ID NO: 7)
MENKTETTVRRRRRIILFPVPFQGHINPILQLANV
LYSKGFSITIFHTNFNKPKTSNYPHFTFRFILDND
PQDERISNLPTHGPLAGMRIPIINEHGADELRREL
ELLMLASEEDEEVSCLITDALWYFAQSVADSLNLR
RLVLMTSSLFNFHAHVSLPQFDELGYLDPDDKTRL
EEQASGFPMLKVKDIKSAYSNWQILKEILGKMIKQ
TKASSGVIWNSFKELEESELETVIREIPAPSFLIP
LPKHLTASSSSLLDHDRTVFQWLDQQPPSSVLYVS
FGSTSEVDEKDFLEIARGLVDSKQSFLWVVRPGFV
KGSTWVEPLPDGFLGERGRIVKWVPQQEVLAHGAI
GAFWTHSGWNSTLESVCEGVPMIFSDFGLDQPLNA
RYMSDVLKVGVYLENGWERGEIANAIRRVMVDEEG
EYIRQNARVLKQKADVSLMKGGSSYESLESLVSYIS
SLGSGANAERMITRVHSQRERLNETLVSERNEVLA
LLSRVEAKGKGILQQNQIIAEFEALPEQTRKKLEG
GPFFDLLKSTQEAIVLPPWVALAVRPRPGVWEYLR
VNLHALVVEELQPAEFLHFKEELVDGVKNGNFTLE
LDFEPFNASIPRPTLHKYIGNGVDFLNRHLSAKLF
HDKESLLPLLKFLRLHSHQGKNLMLSEKIQNLNTL
QHTLRKAEEYLAELKSETLYEEFEAKFEEIGLERG
WGDNAERVLDMIRLLLDLLEAPDPCTLETFLGRVP
MVFNVVILSPHGYFAQDNVLGYPDTGGQVVYILDQ
VRALEIEMLQRIKQQGLNIKPRILILTRLLPDAVG
TTCGERLERVYDSEYCDILRVPFRTEKGIVRKWIS
RFEVWPYLETYTEDAAVELSKELNGKPDLIIGNYS
DGNLVASLLAHKLGVTQCTIAHALEKTKYPDSDIY
WKKLDDKYHFSCQFTADIFAMNHTDFIITSTFQEI
AGSKETVGQYESHTAFTLPGLYRVVHGIDVFDPKF
NIVSPGADMSIYFPYTEEKRRLTKFHSEIEELLYS
DVENKEHLCVLKDKKKPILFTMARLDRVKNLSGLV
EWYGKNTRLRELANLVVVGGDRRKESKDNEEKAEM
KKMYDLIEEYKLNGQFRWISSQMDRVRNGELYRYI
CDTKGAFVQPALYEAFGLTVVEAMTCGLPTFATCK
GGPAEIIVHGKSGFHIDPYHGDQAADTLADFFTKC
KEDPSHWDEISKGGLQRIEEKYTWQIYSQRLLTLT
GVYGFWKHVSNLDRLEARRYLEMFYALKYRPLAQA
VPLAQDDWT
UGT76G1-AtSUS 1 fusion enzyme (GS):
DNA Sequence:
(SEQ ID NO: 8)
ATGGAGAATAAGACAGAAACAACCGTAAGACGGAG
GCGGAGGATTATCTTGTTCCCTGTACCATTTCAGG
GCCATATTAATCCGATCCTCCAATTAGCAAACGTC
CTCTACTCCAAGGGATTTTCAATAACAATCTTCCA
TACTAACTTTAACAAGCCTAAAACGAGTAATTATC
CTCACTTTACATTCAGGTTCATTCTAGACAACGAC
CCTCAGGATGAGCGTATCTCAAATTTACCTACGCA
TGGCCCCTTGGCAGGTATGCGAATACCAATAATCA
ATGAGCATGGAGCCGATGAACTCCGTCGCGAGTTA
GAGCTTCTCATGCTCGCAAGTGAGGAAGACGAGGA
AGTTTCGTGCCTAATAACTGATGCGCTTTGGTACT
TCGCCCAATCAGTCGCAGACTCACTGAATCTACGC
CGTTTGGTCCTTATGACAAGTTCATTATTCAACTT
TCACGCACATGTATCACTGCCGCAATTTGACGAGT
TGGGTTACCTGGACCCGGATGACAAAACGCGATTG
GAGGAACAAGCGTCGGGCTTCCCCATGCTGAAAGT
CAAAGATATTAAGAGCGCTTATAGTAATTGGCAAA
TTCTGAAAGAAATTCTCGGAAAAATGATAAAGCAA
ACCAAAGCGTCCTCTGGAGTAATCTGGAACTCCTT
CAAGGAGTTAGAGGAATCTGAACTTGAAACGGTCA
TCAGAGAAATCCCCGCTCCCTCGTTCTTAATTCCA
CTACCCAAGCACCTTACTGCAAGTAGCAGTTCCCT
CCTAGATCATGACCGAACCGTGTTTCAGTGGCTGG
ATCAGCAACCCCCGTCGTCAGTTCTATATGTAAGC
TTTGGGAGTACTTCGGAAGTGGATGAAAAGGACTT
CTTAGAGATTGCGCGAGGGCTCGTGGATAGCAAAC
AGAGCTTCCTGTGGGTAGTGAGACCGGGATTCGTT
AAGGGCTCGACGTGGGTCGAGCCGTTGCCAGATGG
TTTTCTAGGGGAGAGAGGGAGAATCGTGAAATGGG
TTCCACAGCAAGAGGTTTTGGCTCACGGAGCTATA
GGGGCCTTTTGGACCCACTCTGGTTGGAATTCTAC
TCTTGAAAGTGTCTGTGAAGGCGTTCCAATGATAT
TTTCTGATTTTGGGCTTGACCAGCCTCTAAACGCT
CGCTATATGTCTGATGTGTTGAAGGTTGGCGTGTA
CCTGGAGAATGGTTGGGAAAGGGGGGAAATTGCCA
ACGCCATACGCCGGGTAATGGTGGACGAGGAAGGT
GAGTACATACGTCAGAACGCTCGGGTTTTAAAACA
AAAAGCGGACGTCAGCCTTATGAAGGGAGGTAGCT
CCTATGAATCCCTAGAATCCTTGGTAAGCTATATA
TCTTCGTTAGGTTCTGGTGCAAACGCTGAACGTAT
GATAACGCGCGTCCACAGCCAACGTGAGCGTTTGA
ACGAAACGCTTGTTTCTGAGAGAAACGAAGTCCTT
GCCTTGCTTTCCAGGGTTGAAGCCAAAGGTAAAGG
TATTTTACAACAAAACCAGATCATTGCTGAATTCG
AAGCTTTGCCTGAACAAACCCGGAAGAAACTTGAA
GGTGGTCCTTTCTTTGACCTTCTCAAATCCACTCA
GGAAGCAATTGTGTTGCCACCATGGGTTGCTCTAG
CTGTGAGGCCAAGGCCTGGTGTTTGGGAATACTTA
CGAGTCAATCTCCATGCTCTTGTCGTTGAAGAACT
CCAACCTGCTGAGTTTCTTCATTTCAAGGAAGAAC
TCGTTGATGGAGTTAAGAATGGTAATTTCACTCTT
GAGCTTGATTTCGAGCCATTCAATGCGTCTATCCC
TCGTCCAACACTCCACAAATACATTGGAAATGGTG
TTGACTTCCTTAACCGTCATTTATCGGCTAAGCTC
TTCCATGACAAGGAGAGTTTGCTTCCATTGCTTAA
GTTCCTTCGTCTTCACAGCCACCAGGGCAAGAACC
TGATGTTGAGCGAGAAGATTCAGAACCTCAACACT
CTGCAACACACCTTGAGGAAAGCAGAAGAGTATCT
AGCAGAGCTTAAGTCCGAAACACTGTATGAAGAGT
TTGAGGCCAAGTTTGAGGAGATTGGTCTTGAGAGG
GGATGGGGAGACAATGCAGAGCGTGTCCTTGACAT
GATACGTCTTCTTTTGGACCTTCTTGAGGCGCCTG
ATCCTTGCACTCTTGAGACTTTTCTTGGAAGAGTA
CCAATGGTGTTCAACGTTGTGATCCTCTCTCCACA
TGGTTACTTTGCTCAGGACAATGTTCTTGGTTACC
CTGACACTGGTGGACAGGTTGTTTACATTCTTGAT
CAAGTTCGTGCTCTGGAGATAGAGATGCTTCAACG
TATTAAGCAACAAGGACTCAACATTAAACCAAGGA
TTCTCATTCTAACTCGACTTCTACCTGATGCGGTA
GGAACTACATGCGGTGAACGTCTCGAGAGAGTTTA
TGATTCTGAGTACTGTGATATTCTTCGTGTGCCCT
TCAGAACAGAGAAGGGTATTGTTCGCAAATGGATC
TCAAGGTTCGAAGTCTGGCCATATCTAGAGACTTA
CACCGAGGATGCTGCGGTTGAGCTATCGAAAGAAT
TGAATGGCAAGCCTGACCTTATCATTGGTAACTAC
AGTGATGGAAATCTTGTTGCTTCTTTATTGGCTCA
CAAACTTGGTGTCACTCAGTGTACCATTGCTCATG
CTCTTGAGAAAACAAAGTACCCGGATTCTGATATC
TACTGGAAGAAGCTTGACGACAAGTACCATTTCTC
ATGCCAGTTCACTGCGGATATTTTCGCAATGAACC
ACACTGATTTCATCATCACTAGTACTTTCCAAGAA
ATTGCTGGAAGCAAAGAAACTGTTGGGCAGTATGA
AAGCCACACAGCCTTTACTCTTCCCGGATTGTATC
GAGTTGTTCACGGGATTGATGTGTTTGATCCCAAG
TTCAACATTGTCTCTCCTGGTGCTGATATGAGCAT
CTACTTCCCTTACACAGAGGAGAAGCGTAGATTGA
CTAAGTTCCACTCTGAGATCGAGGAGCTCCTCTAC
AGCGATGTTGAGAACAAAGAGCACTTATGTGTGCT
CAAGGACAAGAAGAAGCCGATTCTCTTCACAATGG
CTAGGCTTGATCGTGTCAAGAACTTGTCAGGTCTT
GTTGAGTGGTACGGGAAGAACACCCGCTTGCGTGA
GCTAGCTAACTTGGTTGTTGTTGGAGGAGACAGGA
GGAAAGAGTCAAAGGACAATGAAGAGAAAGCAGAG
ATGAAGAAAATGTATGATCTCATTGAGGAATACAA
GCTAAACGGTCAGTTCAGGTGGATCTCCTCTCAGA
TGGACCGGGTAAGGAACGGTGAGCTGTACCGGTAC
ATCTGTGACACCAAGGGTGCTTTTGTCCAACCTGC
ATTATATGAAGCCTTTGGGTTAACTGTTGTGGAGG
CTATGACTTGTGGTTTACCGACTTTCGCCACTTGC
AAAGGTGGTCCAGCTGAGATCATTGTGCACGGTAA
ATCGGGTTTCCACATTGACCCTTACCATGGTGATC
AGGCTGCTGATACTCTTGCTGATTTCTTCACCAAG
TGTAAGGAGGATCCATCTCACTGGGATGAGATCTC
AAAAGGAGGGCTTCAGAGGATTGAGGAGAAATACA
CTTGGCAAATCTATTCACAGAGGCTCTTGACATTG
ACTGGTGTGTATGGATTCTGGAAGCATGTCTCGAA
CCTTGACCGTCTTGAGGCTCGCCGTTACCTTGAAA
TGTTCTATGCATTGAAGTATCGCCCATTGGCTCAG
GCTGTTCCTCTTGCACAAGATGATTGA
WT UGT76G1 from Stevia rebaudiana:
Amino Acid Sequence:
(SEQ ID NO: 9)
MENKTETTVRRRRRIILFPVPFQGHINPILQLANV
LYSKGFSITIFHTNFNKPKTSNYPHFTFRFILDND
PQDERISNLPTHGPLAGMRIPIINEHGADELRREL
ELLMLASEEDEEVSCLITDALWYFAQSVADSLNLR
RLVLMTSSLFNFHAHVSLPQFDELGYLDPDDKTRL
EEQASGFPMLKVKDIKSAYSNWQILKEILGKMIKQ
TKASSGVIWNSFKELEESELETVIREIPAPSFLIP
LPKHLTASSSSLLDHDRTVFQWLDQQPPSSVLYVS
FGSTSEVDEKDFLEIARGLVDSKQSFLWVVRPGFV
KGSTWVEPLPDGFLGERGRIVKWVPQQEVLAHGAI
GAFWTHSGWNSTLESVCEGVPMIFSDFGLDQPLNA
RYMSDVLKVGVYLENGWERGEIANAIRRVMVDEEG
EYIRQNARVLKQKADVSLMKGGSSYESLESLVSYI
SSL
WT UGT76G1 from Stevia rebaudiana:
DNA Sequence:
(SEQ ID NO: 10)
ATGGAGAATAAGACAGAAACAACCGTAAGACGGAG
GCGGAGGATTATCTTGTTCCCTGTACCATTTCAGG
GCCATATTAATCCGATCCTCCAATTAGCAAACGTC
CTCTACTCCAAGGGATTTTCAATAACAATCTTCCA
TACTAACTTTAACAAGCCTAAAACGAGTAATTATC
CTCACTTTACATTCAGGTTCATTCTAGACAACGAC
CCTCAGGATGAGCGTATCTCAAATTTACCTACGCA
TGGCCCCTTGGCAGGTATGCGAATACCAATAATCA
ATGAGCATGGAGCCGATGAACTCCGTCGCGAGTTA
GAGCTTCTCATGCTCGCAAGTGAGGAAGACGAGGA
AGTTTCGTGCCTAATAACTGATGCGCTTTGGTACT
TCGCCCAATCAGTCGCAGACTCACTGAATCTACGC
CGTTTGGTCCTTATGACAAGTTCATTATTCAACTT
TCACGCACATGTATCACTGCCGCAATTTGACGAGT
TGGGTTACCTGGACCCGGATGACAAAACGCGATTG
GAGGAACAAGCGTCGGGCTTCCCCATGCTGAAAGT
CAAAGATATTAAGAGCGCTTATAGTAATTGGCAAA
TTCTGAAAGAAATTCTCGGAAAAATGATAAAGCAA
ACCAAAGCGTCCTCTGGAGTAATCTGGAACTCCTT
CAAGGAGTTAGAGGAATCTGAACTTGAAACGGTCA
TCAGAGAAATCCCCGCTCCCTCGTTCTTAATTCCA
CTACCCAAGCACCTTACTGCAAGTAGCAGTTCCCT
CCTAGATCATGACCGAACCGTGTTTCAGTGGCTGG
ATCAGCAACCCCCGTCGTCAGTTCTATATGTAAGC
TTTGGGAGTACTTCGGAAGTGGATGAAAAGGACTT
CTTAGAGATTGCGCGAGGGCTCGTGGATAGCAAAC
AGAGCTTCCTGTGGGTAGTGAGACCGGGATTCGTT
AAGGGCTCGACGTGGGTCGAGCCGTTGCCAGATGG
TTTTCTAGGGGAGAGAGGGAGAATCGTGAAATGGG
TTCCACAGCAAGAGGTTTTGGCTCACGGAGCTATA
GGGGCCTTTTGGACCCACTCTGGTTGGAATTCTAC
TCTTGAAAGTGTCTGTGAAGGCGTTCCAATGATAT
TTTCTGATTTTGGGCTTGACCAGCCTCTAAACGCT
CGCTATATGTCTGATGTGTTGAAGGTTGGCGTGTA
CCTGGAGAATGGTTGGGAAAGGGGGGAAATTGCCA
ACGCCATACGCCGGGTAATGGTGGACGAGGAAGGT
GAGTACATACGTCAGAACGCTCGGGTTTTAAAACA
AAAAGCGGACGTCAGCCTTATGAAGGGAGGTAGCT
CCTATGAATCCCTAGAATCCTTGGTAAGCTATATA
TCTTCGTTATAA
WT AtSUS1 from Arabidopsis thaliana:
Amino Acid Sequence:
(SEQ ID NO: 11)
MANAERMITRVHSQRERLNETLVSERNEVLALLSR
VEAKGKGILQQNQIIAEFEALPEQTRKKLEGGPFF
DLLKSTQEAIVLPPWVALAVRPRPGVWEYLRVNLH
ALVVEELQPAEFLHFKEELVDGVKNGNFTLELDFE
PFNASIPRPTLHKYIGNGVDFLNRHLSAKLFHDKE
SLLPLLKFLRLHSHQGKNLMLSEKIQNLNTLQHTL
RKAEEYLAELKSETLYEEFEAKFEEIGLERGWGDN
AERVLDMIRLLLDLLEAPDPCTLETFLGRVPMVFN
VVILSPHGYFAQDNVLGYPDTGGQVVYILDQVRAL
EIEMLQRIKQQGLNIKPRILILTRLLPDAVGTTCG
ERLERVYDSEYCDILRVPFRTEKGIVRKWISRFEV
WPYLETYTEDAAVELSKELNGKPDLIIGNYSDGNL
VASLLAHKLGVTQCTIAHALEKTKYPDSDIYWKKL
DDKYHFSCQFTADIFAMNHTDFIITSTFQEIAGSK
ETVGQYESHTAFTLPGLYRVVHGIDVFDPKFNIVS
PGADMSIYFPYTEEKRRLTKFHSEIEELLYSDVEN
KEHLCVLKDKKKPILFTMARLDRVKNLSGLVEWYG
KNTRLRELANLVVVGGDRRKESKDNEEKAEMKKMY
DLIEEYKLNGQFRWISSQMDRVRNGELYRYICDTK
GAFVQPALYEAFGLTVVEAMTCGLPTFATCKGGPA
EIIVHGKSGFHIDPYHGDQAADTLADFFTKCKEDP
SHWDEISKGGLQRIEEKYTWQIYSQRLLTLTGVYG
FWKHVSNLDRLEARRYLEMFYALKYRPLAQAVPLA
QDD
WT AtSUS1 from Arabidopsis thaliana:
DNA Sequence:
(SEQ ID NO: 12)
ATGGCAAACGCTGAACGTATGATTACCCGTGTCCA
CTCCCAACGCGAACGCCTGAACGAAACCCTGGTGT
CGGAACGCAACGAAGTTCTGGCACTGCTGAGCCGT
GTGGAAGCTAAGGGCAAAGGTATTCTGCAGCAAAA
CCAGATTATCGCGGAATTTGAAGCCCTGCCGGAAC
AAACCCGCAAAAAGCTGGAAGGCGGTCCGTTTTTC
GATCTGCTGAAATCTACGCAGGAAGCGATCGTTCT
GCCGCCGTGGGTCGCACTGGCAGTGCGTCCGCGTC
CGGGCGTTTGGGAATATCTGCGTGTCAACCTGCAT
GCACTGGTGGTTGAAGAACTGCAGCCGGCTGAATT
TCTGCACTTCAAGGAAGAACTGGTTGACGGCGTCA
AAAACGGTAATTTTACCCTGGAACTGGATTTTGAA
CCGTTCAATGCCAGTATCCCGCGTCCGACGCTGCA
TAAATATATTGGCAACGGTGTGGACTTTCTGAATC
GCCATCTGAGCGCAAAGCTGTTCCACGATAAAGAA
TCTCTGCTGCCGCTGCTGAAATTCCTGCGTCTGCA
TAGTCACCAGGGCAAGAACCTGATGCTGTCCGAAA
AAATTCAGAACCTGAATACCCTGCAACACACGCTG
CGCAAGGCGGAAGAATACCTGGCCGAACTGAAAAG
TGAAACCCTGTACGAAGAATTCGAAGCAAAGTTCG
AAGAAATTGGCCTGGAACGTGGCTGGGGTGACAAT
GCTGAACGTGTTCTGGATATGATCCGTCTGCTGCT
GGACCTGCTGGAAGCACCGGACCCGTGCACCCTGG
AAACGTTTCTGGGTCGCGTGCCGATGGTTTTCAAC
GTCGTGATTCTGTCCCCGCATGGCTATTTTGCACA
GGACAATGTGCTGGGTTACCCGGATACCGGCGGTC
AGGTTGTCTATATTCTGGATCAAGTTCGTGCGCTG
GAAATTGAAATGCTGCAGCGCATCAAGCAGCAAGG
CCTGAACATCAAACCGCGTATTCTGATCCTGACCC
GTCTGCTGCCGGATGCAGTTGGTACCACGTGCGGT
GAACGTCTGGAACGCGTCTATGACAGCGAATACTG
TGATATTCTGCGTGTCCCGTTTCGCACCGAAAAGG
GTATTGTGCGTAAATGGATCAGTCGCTTCGAAGTT
TGGCCGTATCTGGAAACCTACACGGAAGATGCGGC
CGTGGAACTGTCCAAGGAACTGAATGGCAAACCGG
ACCTGATTATCGGCAACTATAGCGATGGTAATCTG
GTCGCATCTCTGCTGGCTCATAAACTGGGTGTGAC
CCAGTGCACGATTGCACACGCTCTGGAAAAGACCA
AATATCCGGATTCAGACATCTACTGGAAAAAGCTG
GATGACAAATATCATTTTTCGTGTCAGTTCACCGC
GGACATTTTTGCCATGAACCACACGGATTTTATTA
TCACCAGTACGTTCCAGGAAATCGCGGGCTCCAAA
GAAACCGTGGGTCAATACGAATCACATACCGCCTT
CACGCTGCCGGGCCTGTATCGTGTGGTTCACGGTA
TCGATGTTTTTGACCCGAAATTCAATATTGTCAGT
CCGGGCGCGGATATGTCCATCTATTTTCCGTACAC
CGAAGAAAAGCGTCGCCTGACGAAATTCCATTCAG
AAATTGAAGAACTGCTGTACTCGGACGTGGAAAAC
AAGGAACACCTGTGTGTTCTGAAAGATAAAAAGAA
ACCGATCCTGTTTACCATGGCCCGTCTGGATCGCG
TGAAGAATCTGTCAGGCCTGGTTGAATGGTATGGT
AAAAACACGCGTCTGCGCGAACTGGCAAATCTGGT
CGTGGTTGGCGGTGACCGTCGCAAGGAATCGAAAG
ATAACGAAGAAAAGGCTGAAATGAAGAAAATGTAC
GATCTGATCGAAGAATACAAGCTGAACGGCCAGTT
TCGTTGGATCAGCTCTCAAATGGACCGTGTGCGCA
ATGGCGAACTGTATCGCTACATTTGCGATACCAAG
GGTGCGTTTGTTCAGCCGGCACTGTACGAAGCTTT
CGGCCTGACCGTCGTGGAAGCCATGACGTGCGGTC
TGCCGACCTTTGCGACGTGTAAAGGCGGTCCGGCC
GAAATTATCGTGCATGGCAAATCTGGTTTCCATAT
CGATCCGTATCACGGTGATCAGGCAGCTGACACCC
TGGCGGATTTCTTTACGAAGTGTAAAGAAGACCCG
TCACACTGGGATGAAATTTCGAAGGGCGGTCTGCA
ACGTATCGAAGAAAAATATACCTGGCAGATTTACA
GCCAACGCCTGCTGACCCTGACGGGCGTCTACGGT
TTTTGGAAACATGTGTCTAATCTGGATCGCCTGGA
AGCCCGTCGCTATCTGGAAATGTTTTACGCACTGA
AGTATCGCCCGCTGGCACAAGCCGTTCCGCTGGCA
CAGGACGACTAA
UGT76G1 L200A mutant (LA)-AtSUS1 fusion
enzyme: Amino Acid Sequence:
(SEQ ID NO: 13)
MENKTETTVRRRRRIILFPVPFQGHINPILQLANV
LYSKGFSITIFHTNFNKPKTSNYPHFTFRFILDND
PQDERISNLPTHGPLAGMRIPIINEHGADELRREL
ELLMLASEEDEEVSCLITDALWYFAQSVADSLNLR
RLVLMTSSLFNFHAHVSLPQFDELGYLDPDDKTRL
EEQASGFPMLKVKDIKSAYSNWQIAKEILGKMIKQ
TKASSGVIWNSFKELEESELETVIREIPAPSFLIP
LPKHLTASSSSLLDHDRTVFQWLDQQPPSSVLYVS
FGSTSEVDEKDFLEIARGLVDSKQSFLWVVRPGFV
KGSTWVEPLPDGFLGERGRIVKWVPQQEVLAHGAI
GAFWTHSGWNSTLESVCEGVPMIFSDFGLDQPLNA
RYMSDVLKVGVYLENGWERGEIANAIRRVMVDEEG
EYIRQNARVLKQKADVSLMKGGSSYESLESLVSYI
SSLGSGANAERMITRVHSQRERLNETLVSERNEVL
ALLSRVEAKGKGILQQNQIIAEFEALPEQTRKKLE
GGPFFDLLKSTQEAIVLPPWVALAVRPRPGVWEYL
RVNLHALVVEELQPAEFLHFKEELVDGVKNGNFTL
ELDFEPFNASIPRPTLHKYIGNGVDFLNRHLSAKL
FHDKESLLPLLKFLRLHSHQGKNLMLSEKIQNLNT
LQHTLRKAEEYLAELKSETLYEEFEAKFEEIGLER
GWGDNAERVLDMIRLLLDLLEAPDPCTLETFLGRV
PMVFNVVILSPHG
YFAQDNVLGYPDTGGQVVYILDQVRALEIEMLQRI
KQQGLNIKPRILILTRLLPDAVGTTCGERLERVYD
SEYCDILRVPFRTEKGIVRKWISRFEVWPYLETYT
EDAAVELSKELNGKPDLIIGNYSDGNLVASLLAHK
LGVTQCTIAHALEKTKYPDSDIYWKKLDDKYHFSC
QFTADIFAMNHTDFIITSTFQEIAGSKETVGQYES
HTAFTLPGLYRVVHGIDVFDPKFNIVSPGADMSIY
FPYTEEKRRLTKFHSEIEELLYSDVENKEHLCVLK
DKKKPILFTMARLDRVKNLSGLVEWYGKNTRLREL
ANLVVVGGDRRKESKDNEEKAEMKKMYDLIEEYKL
NGQFRWISSQMDRVRNGELYRYICDTKGAFVQPAL
YEAFGLTVVEAMTCGLPTFATCKGGPAEIIVHGKS
GFHIDPYHGDQAADTLADFFTKCKEDPSHWDEISK
GGLQRIEEKYTWQIYSQRLLTLTGVYGFWKHVSNL
DRLEARRYLEMFYALKYRPLAQAVPLAQDDWT
UGT76G1 L200A mutant (LA)-AtSUS1
fusion enzyme: DNA Sequence:
(SEQ ID NO: 14)
ATGGAGAATAAGACAGAAACAACCGTAAGACGGAG
GCGGAGGATTATCTTGTTCCCTGTACCATTTCAGG
GCCATATTAATCCGATCCTCCAATTAGCAAACGTC
CTCTACTCCAAGGGATTTTCAATAACAATCTTCCA
TACTAACTTTAACAAGCCTAAAACGAGTAATTATC
CTCACTTTACATTCAGGTTCATTCTAGACAACGAC
CCTCAGGATGAGCGTATCTCAAATTTACCTACGCA
TGGCCCCTTGGCAGGTATGCGAATACCAATAATCA
ATGAGCATGGAGCCGATGAACTCCGTCGCGAGTTA
GAGCTTCTCATGCTCGCAAGTGAGGAAGACGAGGA
AGTTTCGTGCCTAATAACTGATGCGCTTTGGTACT
TCGCCCAATCAGTCGCAGACTCACTGAATCTACGC
CGTTTGGTCCTTATGACAAGTTCATTATTCAACTT
TCACGCACATGTATCACTGCCGCAATTTGACGAGT
TGGGTTACCTGGACCCGGATGACAAAACGCGATTG
GAGGAACAAGCGTCGGGCTTCCCCATGCTGAAAGT
CAAAGATATTAAGAGCGCTTATAGTAATTGGCAAA
TTGCGAAAGAAATTCTCGGAAAAATGATAAAGCAA
ACCAAAGCGTCCTCTGGAGTAATCTGGAACTCCTT
CAAGGAGTTAGAGGAATCTGAACTTGAAACGGTCA
TCAGAGAAATCCCCGCTCCCTCGTTCTTAATTCCA
CTACCCAAGCACCTTACTGCAAGTAGCAGTTCCCT
CCTAGATCATGACCGAACCGTGTTTCAGTGGCTGG
ATCAGCAACCCCCGTCGTCAGTTCTATATGTAAGC
TTTGGGAGTACTTCGGAAGTGGATGAAAAGGACTT
CTTAGAGATTGCGCGAGGGCTCGTGGATAGCAAAC
AGAGCTTCCTGTGGGTAGTGAGACCGGGATTCGTT
AAGGGCTCGACGTGGGTCGAGCCGTTGCCAGATGG
TTTTCTAGGGGAGAGAGGGAGAATCGTGAAATGGG
TTCCACAGCAAGAGGTTTTGGCTCACGGAGCTATA
GGGGCCTTTTGGACCCACTCTGGTTGGAATTCTAC
TCTTGAAAGTGTCTGTGAAGGCGTTCCAATGATAT
TTTCTGATTTTGGGCTTGACCAGCCTCTAAACGCT
CGCTATATGTCTGATGTGTTGAAGGTTGGCGTGTA
CCTGGAGAATGGTTGGGAAAGGGGGGAAATTGCCA
ACGCCATACGCCGGGTAATGGTGGACGAGGAAGGT
GAGTACATACGTCAGAACGCTCGGGTTTTAAAACA
AAAAGCGGACGTCAGCCTTATGAAGGGAGGTAGCT
CCTATGAATCCCTAGAATCCTTGGTAAGCTATATA
TCTTCGTTAGGTTCTGGTGCAAACGCTGAACGTAT
GATAACGCGCGTCCACAGCCAACGTGAGCGTTTGA
ACGAAACGCTTGTTTCTGAGAGAAACGAAGTCCTT
GCCTTGCTTTCCAGGGTTGAAGCCAAAGGTAAAGG
TATTTTACAACAAAACCAGATCATTGCTGAATTCG
AAGCTTTGCCTGAACAAACCCGGAAGAAACTTGAA
GGTGGTCCTTTCTTTGACCTTCTCAAATCCACTCA
GGAAGCAATTGTGTTGCCACCATGGGTTGCTCTAG
CTGTGAGGCCAAGGCCTGGTGTTTGGGAATACTTA
CGAGTCAATCTCCATGCTCTTGTCGTTGAAGAACT
CCAACCTGCTGAGTTTCTTCATTTCAAGGAAGAAC
TCGTTGATGGAGTTAAGAATGGTAATTTCACTCTT
GAGCTTGATTTCGAGCCATTCAATGCGTCTATCCC
TCGTCCAACACTCCACAAATACATTGGAAATGGTG
TTGACTTCCTTAACCGTCATTTATCGGCTAAGCTC
TTCCATGACAAGGAGAGTTTGCTTCCATTGCTTAA
GTTCCTTCGTCTTCACAGCCACCAGGGCAAGAACC
TGATGTTGAGCGAGAAGATTCAGAACCTCAACACT
CTGCAACACACCTTGAGGAAAGCAGAAGAGTATCT
AGCAGAGCTTAAGTCCGAAACACTGTATGAAGAGT
TTGAGGCCAAGTTTGAGGAGATTGGTCTTGAGAGG
GGATGGGGAGACAATGCAGAGCGTGTCCTTGACAT
GATACGTCTTCTTTTGGACCTTCTTGAGGCGCCTG
ATCCTTGCACTCTTGAGACTTTTCTTGGAAGAGTA
CCAATGGTGTTCAACGTTGTGATCCTCTCTCCACA
TGGTTACTTTGCTCAGGACAATGTTCTTGGTTACC
CTGACACTGGTGGACAGGTTGTTTACATTCTTGAT
CAAGTTCGTGCTCTGGAGATAGAGATGCTTCAACG
TATTAAGCAACAAGGACTCAACATTAAACCAAGGA
TTCTCATTCTAACTCGACTTCTACCTGATGCGGTA
GGAACTACATGCGGTGAACGTCTCGAGAGAGTTTA
TGATTCTGAGTACTGTGATATTCTTCGTGTGCCCT
TCAGAACAGAGAAGGGTATTGTTCGCAAATGGATC
TCAAGGTTCGAAGTCTGGCCATATCTAGAGACTTA
CACCGAGGATGCTGCGGTTGAGCTATCGAAAGAAT
TGAATGGCAAGCCTGACCTTATCATTGGTAACTAC
AGTGATGGAAATCTTGTTGCTTCTTTATTGGCTCA
CAAACTTGGTGTCACTCAGTGTACCATTGCTCATG
CTCTTGAGAAAACAAAGTACCCGGATTCTGATATC
TACTGGAAGAAGCTTGACGACAAGTACCATTTCTC
ATGCCAGTTCACTGCGGATATTTTCGCAATGAACC
ACACTGATTTCATCATCACTAGTACTTTCCAAGAA
ATTGCTGGAAGCAAAGAAACTGTTGGGCAGTATGA
AAGCCACACAGCCTTTACTCTTCCCGGATTGTATC
GAGTTGTTCACGGGATTGATGTGTTTGATCCCAAG
TTCAACATTGTCTCTCCTGGTGCTGATATGAGCAT
CTACTTCCCTTACACAGAGGAGAAGCGTAGATTGA
CTAAGTTCCACTCTGAGATCGAGGAGCTCCTCTAC
AGCGATGTTGAGAACAAAGAGCACTTATGTGTGCT
CAAGGACAAGAAGAAGCCGATTCTCTTCACAATGG
CTAGGCTTGATCGTGTCAAGAACTTGTCAGGTCTT
GTTGAGTGGTACGGGAAGAACACCCGCTTGCGTGA
GCTAGCTAACTTGGTTGTTGTTGGAGGAGACAGGA
GGAAAGAGTCAAAGGACAATGAAGAGAAAGCAGAG
ATGAAGAAAATGTATGATCTCATTGAGGAATACAA
GCTAAACGGTCAGTTCAGGTGGATCTCCTCTCAGA
TGGACCGGGTAAGGAACGGTGAGCTGTACCGGTAC
ATCTGTGACACCAAGGGTGCTTTTGTCCAACCTGC
ATTATATGAAGCCTTTGGGTTAACTGTTGTGGAGG
CTATGACTTGTGGTTTACCGACTTTCGCCACTTGC
AAAGGTGGTCCAGCTGAGATCATTGTGCACGGTAA
ATCGGGTTTCCACATTGACCCTTACCATGGTGATC
AGGCTGCTGATACTCTTGCTGATTTCTTCACCAAG
TGTAAGGAGGATCCATCTCACTGGGATGAGATCTC
AAAAGGAGGGCTTCAGAGGATTGAGGAGAAATACA
CTTGGCAAATCTATTCACAGAGGCTCTTGACATTG
ACTGGTGTGTATGGATTCTGGAAGCATGTCTCGAA
CCTTGACCGTCTTGAGGCTCGCCGTTACCTTGAAA
TGTTCTATGCATTGAAGTATCGCCCATTGGCTCAG
GCTGTTCCTCTTGCACAAGATGATTGA

What is claimed is:

1. A method for synthesizing rebaudioside I, the method comprising preparing a reaction mixture comprising:
(a) a steviol glycoside composition comprising rebaudioside A;
(b) a substrate selected from the group consisting of sucrose, uridine diphosphate (UDP), uridine diphosphate-glucose (UDP-glucose), and combinations thereof; and
(c) a UDP-glycosyltransferase enzyme comprising the amino acid sequence of SEQ ID NO: 1; and
incubating the reaction mixture for a sufficient time to produce rebaudioside I.

2. The method of claim 1, wherein the steviol glycoside composition is stevia extract.

3. The method of claim 1, further comprising adding a sucrose synthase to the reaction mixture.

4. The method of claim 3, wherein the sucrose synthase is an Arabidopsis thaliana sucrose synthase 1 (AtSUS1) comprising the amino acid sequence of SEQ ID NO: 11.

5. The method of claim 1, wherein the reaction mixture is in vitro.

6. The method of claim 1. wherein the reaction mixture is a cell-based reaction mixture.

7. The method of claim 6, wherein the UDP-glycosyltransferase enzyme is expressed in a host cell.

8. The method of claim 7, wherein the host cell is selected from the group consisting of a yeast, a non-steviol glycoside producing plant, an alga, a fungus, and a bacterium.

9. The method of claim 7, wherein the host cell is a bacterial cell.

10. The method of claim 9, wherein the bacterial cell is an E. coli cell.

11. The method of claim 7, wherein the host cell is a yeast cell.

12. The method of claim 1, wherein the subject is UDP-glucose.

13. The method of claim 1, wherein the rebaudioside A has a concentration of 15 to 50 g/L in the reaction mixture.

14. The method to claim 1, wherein the reaction mixture has a pH range of 6.5 to 9.5 at a temperature of 35° C. to 45° C.

15. The method of claim 1, further comprising isolating crude rebaudioside I.

16. The method of claim 15, further comprising crystallizing the crude rebaudioside Ito obtain rebaudioside I with a purity of greater than 98%.

17. A UGT76G1 mutant comprising a L200A mutation relative to SEQ ID NO: 9.

18. The UGT76G1 mutant of claim 17, comprising the amino acid sequence of SEQ ID NO: 1.
